(19) United States
(12) Patent Application Publication
(10) Pub. No.: US 2019/0015519 A1
(43) Pub. Date: Jan. 17, 2019

(54) MMP-SENSITIVE TAXANE PRODRUG

(71) Applicant: Incanthera Ltd, Manchester (GB)

(72) Inventors: Paul Loadman, Bradford (GB); Robert Falconer, West Yorkshire (GB); Jason Gill, Leeds (GB)

(21) Appl. No.: 16/065,517

(22) PCT Filed: Nov. 29, 2016
(86) PCT No.: PCT/GB2016/053745

§ 371 Date: Jun. 22, 2018

(30) Foreign Application Priority Data
Dec. 1, 2015 (GB) 1521215.2

(51) Int. Cl. A61K 47/64; A61K 47/65; A61K 31/337; A61P 35/00

(52) U.S. Cl. CPC A61K 47/64; A61P 35/00; A61K 31/337; A61K 47/65

(57) ABSTRACT

The present invention harnesses the differential expression of membrane-type matrix metalloproteinases (MT-MMPs) between human solid tumours and normal tissues to provide a systemically inactive prodrug which is selectively activated at the tumour micro-environment. The present invention provides a prodrug which is a conjugate of a taxane and a selective MT-MMP cleavable delivery vehicle.

### INTRODUCTION

Paclitaxel (Taxol) is a cytoskeletal drug of the taxane class that targets 62 -tubulin and stabilizes microtubules. Paclitaxel-treated cells have defects in mitotic spindle assembly, chromosome segregation, and cell division. This blocks progression of mitosis and therefore paclitaxel is used in chemotherapy. Paclitaxel therapy is approved for the treatment of a wide range of solid tumours.

Administration of paclitaxel to a patient also leads to stabilization of microtubules in non-cancerous cells and this causes significant off-target toxicities and side effects. The key dose-limiting systemic toxicities associated with paclitaxel administration are myelotoxicity, neurotoxicity and hepatotoxicity. Common side effects include nausea and vomiting, loss of appetite, change in taste, thinned or brittle hair, pain in the joints of the arms or legs lasting two to three days, changes in the colour of the nails, and tingling in the hands or toes. More serious side effects such as unusual bruising or bleeding, pain/redness/swelling at the injection site, Hand-foot syndrome, change in normal bowel habits for more than two days, fever, chills, cough, sore throat, difficulty swallowing, dizziness, shortness of breath, severe exhaustion, skin rash, facial flushing, female infertility by ovarian damage and chest pain can also occur.

The clinical utility of taxanes such as paclitaxel is restricted by their toxicity towards healthy cells, resulting in a narrow therapeutic index and subsequent reduction in treatment benefit. It would be advantageous to target paclitaxel selectively to the tumour and consequently reduce normal tissue toxicity and side effects. One means of approaching this objective is the design of prodrug molecules that are specifically targeted to or selectively activated in tumour tissue, thereby reducing systemic levels of paclitaxel and increasing the therapeutic index.

Research is ongoing to find a way to mitigate the side effects of paclitaxel by altering its administration with the aim that the cytotoxic effects are experienced only or to a greater degree by tumour cells than by normal cells. There remains a need to find a tumour-activated paclitaxel prodrug that is systemically safe and yet is activated at the tumour microenvironment where the paclitaxel can have its cytotoxic effects.

### SUMMARY

Membrane-type matrix metalloproteinases (MT-MMPs) are highly elevated in human solid tumours, where they are central to tumour invasion and angiogenesis. In contrast MT-MMPs are absent or inactive in normal tissues.

The present invention harnesses this difference in MT-MMP expression as the key to providing a systemically inactive prodrug which is selectively activated at the tumour micro-environment. Therefore the present invention provides a prodrug which is a conjugate of a taxane and a selective MT-MMP cleavable delivery vehicle.

In a first aspect, the present invention provides a conjugate comprising a taxane linked directly or indirectly via a self-immolative linker to a peptide comprising a membrane type matrix metalloproteinase (MT-MMP) recognition sequence and cleavage site having the amino acid sequence -Arg-Ser-aa1-Gly-Hof-aa2-aa3-, wherein each of aa1, aa2 and aa3 is any amino acid.

In a second aspect, the present invention provides a conjugate comprising paclitaxel linked directly or indirectly via a para-amino benzoic acid (PAB) linker moiety to a peptide comprising a membrane type matrix metalloproteinase (MT-MMP) recognition sequence and cleavage site having the amino acid sequence -Arg-Ser-Cit-Gly-Hof-Tyr-Leu-, and having an FITC capping group.

In a third aspect, the present invention provides a pharmaceutical formulation comprising a conjugate according to the first or second aspect of this invention and a pharmaceutically acceptable carrier, diluent or excipient.

In a fourth aspect a conjugate of the invention is for use in medicine. Additionally a conjugate of the invention is for use in treating cancer. Additionally, the present invention provides a method of treating a cancer in a subject, comprising administering a therapeutically effective amount of conjugate of the present invention to a subject in need thereof. In particular the cancer is a prostate cancer.

### DESCRIPTION OF THE FIGURES

FIG. 1 shows a schematic representation of a conjugate of the present invention in which the oval shape is a capping group, brown blocks represent amino acids, the blue block represents a self-immolative linker, and dark blue shape labelled T is a taxane that is not active and cannot exert its cytotoxic activity (due to conjugation to the self-immolative linker and peptide). FIG. 1A shows the conjugate and indicates the site at which an MT-MMP can cleave the MT-MMP cleavable amino acid sequence. FIG. 1B shows the conjugate after cleavage by a MT-MMP. The taxane remains inactive. Action of non-specific peptidases in the tumour microenvironment sequentially and rapidly remove amino acids until only the inactive taxane linked to the self-immolative linker remains as shown in FIG. 1C. The self-immolative linker rapidly and spontaneously cleaves to release active taxane as shown in FIG. 1D.

FIG. 2 shows schematics of a number of conjugates.

FIG. 3 shows the chemical structure and a schematic of ICT3205, which is a conjugate comprising paclitaxel linked via a para-amino benzoic acid (PAB) linker moiety to a peptide comprising a membrane type matrix metalloproteinase (MT-MMP) cleavage site having the amino acid sequence -Arg-Ser-Cit-Gly-Hof-Tyr-Leu-, and having an FITC capping group. ICT3205 has chemical formula: C121H₁₃₆N₁₆O₃₂S, exact mass: 2356.92 and molecular weight: 2358.53.

FIG. 4 shows the predicted cleavage of ICT3205 by MMP-14 or by MMP-15.

FIG. 5 shows traces of the cleavage of ICT3205 by recombinant proteins in vitro (MMP14 top two, MMP 15 bottom two).

The parent at 9.16 min is cleaved to produce the FITC N-terminal peptide fragment (FITC half) at 1.39 min over 18 h and the C-terminal peptide fragment (paclitaxel half) at 9.16 min.

This is confirmed in the next slide by mass spectrometry.

FIG. 6 shows confirmation of cleavage is shown by mass spectrometry Bottom two traces show the fragment (m/z721.3) appears together with the fragment m/z937 (top two traces) following incubation with MMP14.

FIG. 7 shows a summary of metabolic data both graphically and in tabular form Parent molecules are incubated with tissue homogenate (tumour tissue activation and normal tissue stability) and half lives calculated

Green indicates acceptable whereas red is unacceptable.

All incubations carried out at lum (whereas in the next Figure they are at 10 uM)

FIG. 8 shows a summary of metabolic data both graphically and in tabular form

Parent molecules are incubated with tissue homogenate and half lives calculated Green indicates acceptable whereas red is unacceptable.

All incubations carried out at 10 um (whereas the previous Figure they are at 1uM).

FIG. 9 shows a summary of metabolic data both graphically and in tabular form Parent molecules are incubated in tissue culture media containing the cells described and half lives calculated

Green indicates acceptable whereas red is unacceptable.

All incubations carried out at 20 um. ICT3205 shows superior cleavage by prostate tumour-associated MT-MMPs and release of paclitaxel compared to other agents tested. ICT3205 additionally shows stability (and lack of activation and paclitaxel release) in normal prostate cells (NT2).

FIG. 10 shows incubation of drugs in kidney and liver homogenates showing the marked increased stability of ICT3205 over other molecules,

FIG. 11 shows a summary table of drug stability in kidney and liver and demonstrates the increased stability of ICT3205 over other molecules.

FIG. 12 shows a summary of the summary tables but demonstrating that conjugates ICT3179 and ICT3199 are less preferred.

FIG. 13 shows a summary of the summary tables and demonstrates that ICT3205 has uniquely and unexpectedly advantageous properties of specificity and selectivity in comparison with other tested conjugates and therefore is the preferred molecule to take forward to in vivo studies.

FIG. 14 shows a further summary of the summary tables but demonstrating that ICT3205 is the preferred molecule but with data omitted and simplifying to a colour scheme.

FIG. 15 shows a summary graph confirming that ICT3205 is the most preferred conjugate.

Whether you examine normal tissue stability or tumour activation (shown as tumour stability) or cell stability ICT3205 is always the compound of choice.

FIG. 16 shows a comparison of paclitaxel conjugates in either liver homogenate or prostate cancer cells line PC3 homogenate. This demonstrates conjugate stability in liver and conjugate activation and paclitaxel release in PC3 tumour cells. The traces demonstrate the disappearance (i.e. activation) of ICT3205 in the tumour (right) and relative stability in the liver (left) and the appearance of paclitaxel (Taxol) in the tumour but not in the liver. A paclitaxel (Taxol) standard is shown in the top trace,

FIG. 17 shows that ICT3205 is metabolised equally quickly in either PC3 or LNCaP prostate cancer cell lines.

FIG. 18 shows that ICT3205 is metabolised rapidly in PC3 but stable in normal tissue homogenates (i.e. liver, kidney, lung)

FIG. 19 shows that ICT3205 conjugate is metabolised in PC3 and LNCaP prostate cancer cell lines and has a long in vitro ‘half-life’ and stability in normal tissues liver, kidney and lung.

FIG. 20 shows the results of a pharmacokinetics study following administration of paclitaxel alone (blue lines) and following administration of ICT3205 (equimolar doses). The yellow line shows paclitaxel release from the conjugate (the green line shows parent ICT3205). The early peak drug concentrations associated with paclitaxel dosing are removed by administration of prodrug. A 10-fold increase in tumour concentration of paclitaxel is observed following administration as ICT3205.

FIG. 21 provides a table highlighting in greater detail the disparity of distribution between normal tissues and tumour tissues for paclitaxel alone, parent ICT3205 and paclitaxel release from the prodrug. Levels of paclitaxel released from ICT3205 were consistently below detectable levels in plasma and <0.5 uM in heart strongly implicating the potential of ICT3205 for decreased systemic toxicity.

FIG. 22 shows superior anti-tumour effects of conjugate ICT3205 of the present invention when tested in vivo in tumour (PC3)-bearing mouse studies, as compared to the equivalent molar dose of paclitaxel, which demonstrates no efficacy at the concentrations administered.

FIG. 23 shows that there is no toxicity and no adverse systemic effects from administration of conjugate ICT3205 to mice, as indicated by mouse body weight.

FIG. 24 shows how a self-immolative linker spontaneously removes itself to release active paclitaxel.

### DESCRIPTION

Taxan/Paclitaxel

The present invention provides a taxane prodrug. The prodrug of the present invention overcomes the toxic effects of systemic administration of a taxane and is activated at the tumour micro-environment to release the taxane. Therefore the present invention provides a prodrug which is a conjugate of a taxane and a MT-MMP cleavable delivery vehicle.

Taxanes are anti-tumour agents and are a group of natural products (Taxus sp.) or analogues, which are diterpenes with a taxadiene core. Taxanes bind to and stabilize microtubules causing cell-cycle arrest and apoptosis (cell death). At the tumour micro-environment taxanes can inhibit tumour cell division and inhibit cell division of endothelial cells forming neovasculature required for the survival of tumour cells.

Paclitaxel (also known Taxol), initially extracted from the bark of the Pacific yew tree, Taxus brevifolia, was identified in 1971 as part of a National Cancer Institute (NCI) program that screened medicinal plants for potential activity. Docetaxel (also known as Taxotere) was synthesized in 1986, using a precursor extracted from the needles of the European yew, T. baccata, and is similar to paclitaxel in its mechanism of action. In 2010 another Taxane, cabazitaxel was FDA approved in cancer therapy.

A conjugate of the present invention can comprise a taxane. Optionally a conjugate of the present invention comprises paclitaxel, docetaxel or cabazitaxel. Preferably a conjugate of the present invention comprises paclitaxel.

MT-MMPs

A conjugate of the present invention comprises a peptide cleavable by a MT-MMP. Matrix metalloproteinases (MMPs) degrade extracellular matrix proteins and play an important role in tissue remodelling. Membrane type matrix metalloproteinases (MT-MMPs) are transmembrane proteins and include MMP-14 (MT1-MMP), MMP-15 (MT2-MMP), MMP-16 (MT3-MMP) and MMP17 (MT4-MMP). MT-MMPs are highly elevated at the solid tumour micro-environment where they play a role in angiogenesis and development of tumour neovasculature, tumour growth and invasion into surrounding tissues. MMP-14 and MMP-15 are highly elevated at the site of a solid tumour and at the microenvironment of a solid tumour.

Analysis was performed of the difference in expression of MMPs between prostate cancer cells lines and normal (non-tumour) tissues. It was determined that the expression of both mRNA and protein for MMP-14 and/or MMP-15 in prostate cancer cells lines is many orders of magnitude greater in prostate cancer cells than in normal tissues. MMP-14 and/or MMP-15 play an important role in the development of prostate cancer. Furthermore MMP-15 expression in tissue or serum has been positively correlated with Gleason score in prostate cancer.

The Peptide Comprising the MT-MMP Cleavable Site

The peptide comprises a MT-MMP cleavage site having the amino acid sequence -Arg-Ser-aa1-Gly-Hof-aa2-aa3-, wherein each of aa1, aa2 and aa3 can be any amino acid.

In the MT-MMP cleavage site amino acid aal can be selected from Cit and Arg, and preferably aa1 is Cit. The amino acid aa2 can be selected from Tyr, Asp, Ala, Ser, Asn, Pro, Leu, and preferably aa2 is Tyr. The amino acid aa3 can be selected from Leu and Asn, and preferably aa3 is Leu. The MT-MMP cleavage site can have an amino acid sequence selected from
-Arg-Ser-Cit-Gly-Hof-Tyr-Leu-,
-Arg-Ser-Cit-Gly-Hof-Asn-Tyr-, or
-Arg-Ser-Arg-Gly-Hof-Tyr-Leu-.

Preferably the MT-MMP cleavage site has the amino acid sequence -Arg-Ser-Cit-Gly-Hof-Tyr-Leu-.

The peptide can comprise one or more amino acids at one or both ends of the MT-MMP cleavage site.

Capping Groups

The conjugate can have a capping group on the peptide to prevent non-specific degradation of the peptide. The capping group can be at the end of the peptide which is not linked directly or indirectly via a linker moiety to a taxane. The capping group can include any appropriate group at the N- or C-terminus of the peptide. The capping group can be selected from simple sugars, D-amino acids, proline amino acids, aromatics, aliphatics, fluorescein, fluorescein derivatives (advantageously from fluorescein isothiocycante (FITC)), polyethylene glycol (PEG) and derivatives. Preferably a conjugate of the present invention has a fluorescein capping group resulting from coupling fluorescein isothiocyanate (FITC) to the N-terminal amino acid.

Self-Immolative Linker

Subsequent to MT-MMP action at the tumour microenvironment to cleave the MT-MMP cleavage site, activity of ubiquitous exopeptidases rapidly removes each newly exposed terminal amino acid. However, the present inventors have identified that the activity of ubiquitous exopeptidases is not able to efficiently cleave an amino acid from a taxane, Also, an ester linkage, whilst cleavable, is not preferred in terms of normal tissue stability of the conjugate. Therefore an amino acid or other linker moiety which is not self immolative may require enzyme action to remove it from the taxane. The present inventors have identified that enzymes cannot readily cleave linkers which are not self-immolative linkers from a taxane. Taxanes linked to an amino acid or other molecule are not active.

A conjugate of the present invention advantageously comprises a self-immolative linker between the peptide cleavable by a MT-MMP and the taxane. The taxane is linked, directly or indirectly, via the self-immolative linker to the peptide comprising a MT-MMP cleavage site. A self-immolative linker is chemically unstable once the terminal amino acid has been metabolised by exopeptidases. A self-immolative linker is capable of undergoing a spontaneous intramolecular reaction to rapidly release the taxane in its parent, active form.

Therefore a conjugate of the present invention having a self-immolative linker between the taxane and the peptide comprising a MT-MMP cleavage site has improved release of the cytotoxic taxane at the tumour micro-environment. Taxane release from a conjugate of the present invention at the tumour microenvironment is rapid.

Preferably the self-immolative linker is para-amino benzoic acid (PAB).

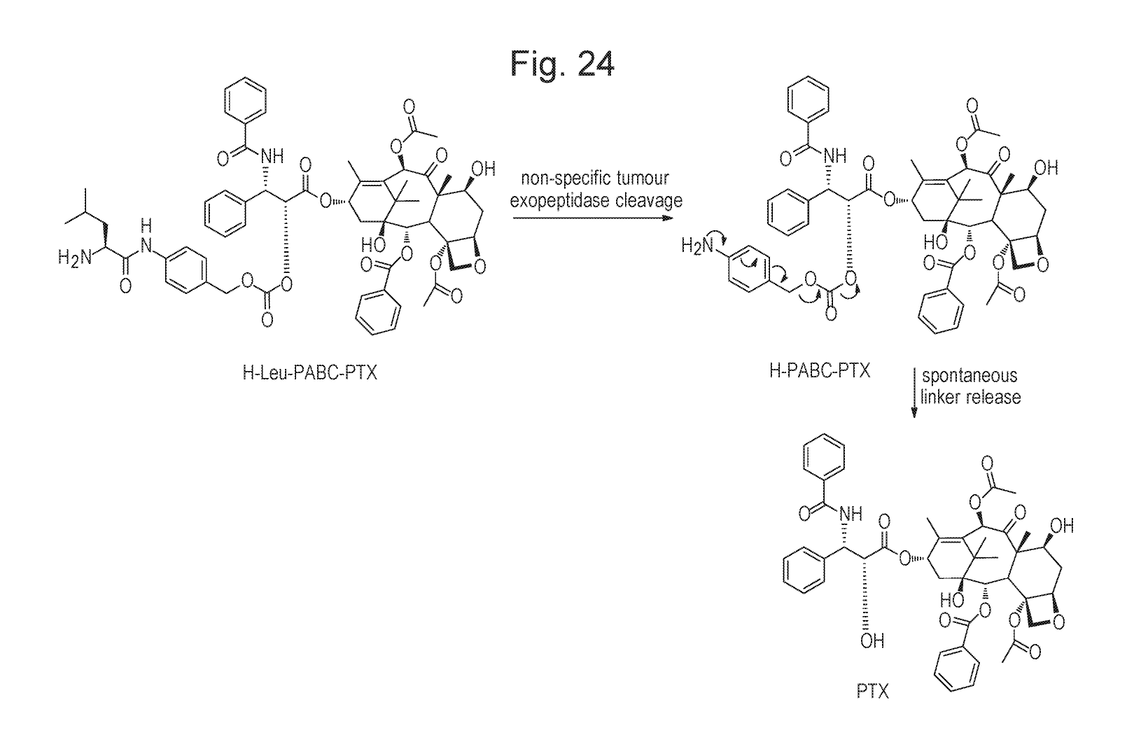

Spontaneous cleavage of a self-immolative linker is shown in FIG. 24.

Spacers

A conjugate of the present invention can comprise a spacer between the peptide and the capping group. A conjugate of the present invention can comprise a spacer between the self-immolative linker and the peptide. The spacer can selected from an amino acid, a non-natural amino acid, optionally n-Ala or a succinyl group. Preferably a conjugate of the present invention has a 3-Ala spacer between the peptide and the capping group.

Conjugate Structures

Preferably a conjugate of the present invention has the taxane is linked directly or indirectly via the self-immolative linker to the C-terminus of the peptide. Advantageously, a conjugate of the present invention has the capping group at the N-terminus of the peptide.

In embodiments a conjugate of the present invention comprises paclitaxel linked via PAB to the C-terminus of the peptide comprising -Arg-Ser-Cit-Gly-Hof-Tyr-Leu- and having the capping group FITC group at the N-terminus and having the spacer group beta-Ala between the peptide and the capping group.

A conjugate of the present invention can have a structure comprising Y-L-X, wherein Y is a peptide comprising a membrane type matrix metalloproteinase (MT-MMP) peptide recognition sequence, L is a self-immolative linker and X is a taxane, each of the peptide, self-immolative linker and taxane are as described above,

Optionally a conjugate of the present invention can have a structure comprising C-Y-L-X, wherein Y is a peptide comprising a membrane type matrix metalloproteinase (MT-MMP) L is a self-immolative linker, X is a taxane and C is a capping group, each of the peptide, self-immolative linker, taxane and capping group are as described above.

Further optionally a conjugate of the present invention can have a structure comprising C-a-Y-L-X or comprising C-a-Y-a-L-X, wherein Y is a peptide comprising a membrane type matrix metalloproteinase (MT-MMP) L is a self-immolative linker, X is a taxane, C is a capping group and a is a linker, each of the peptide, self-immolative linker, taxane, capping group and spacer are as described above.

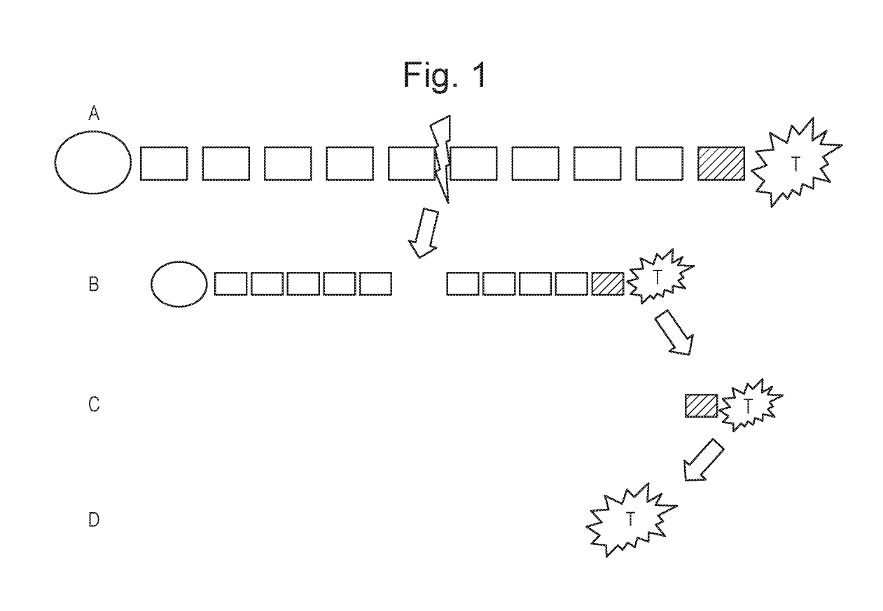

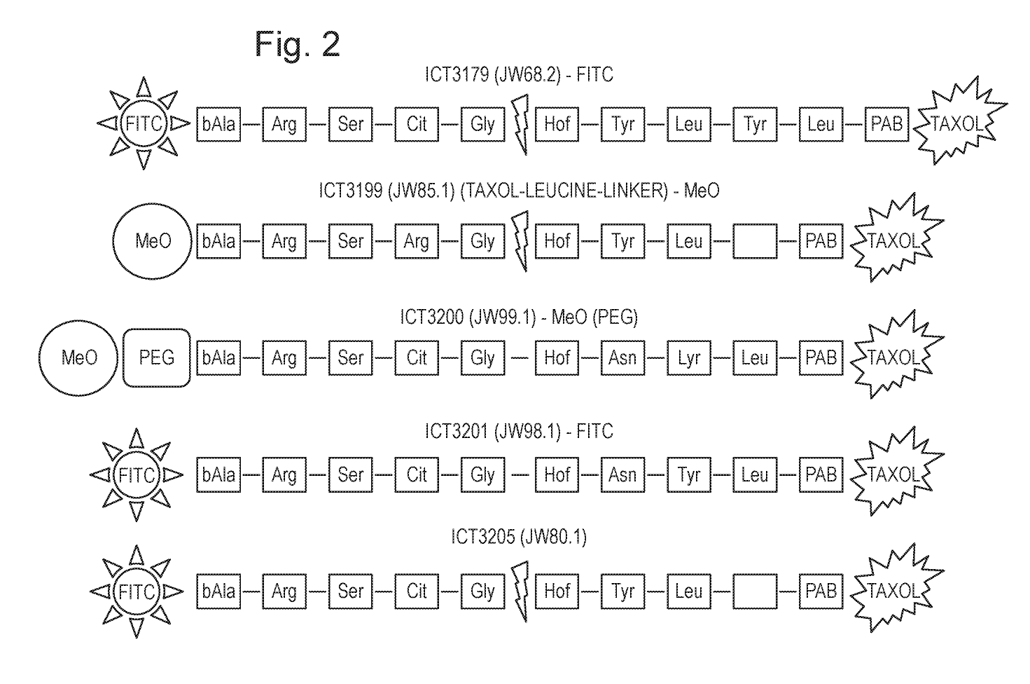

The general structure of a conjugate of the present invention is shown in FIG. 1A which indicates the MT-MMP cleavage site in the peptide, FIGS. 1B-D show how MT-MMP enzyme action initiates the release of active taxane. FIG. 2 shows five conjugates of the present invention.

ICT3205

Conjugates of the present invention were analysed for stability in normal tissues and for speed and extent of release of active cytotoxic taxane in tumour tissues. One conjugate demonstrated uniquely superior properties.

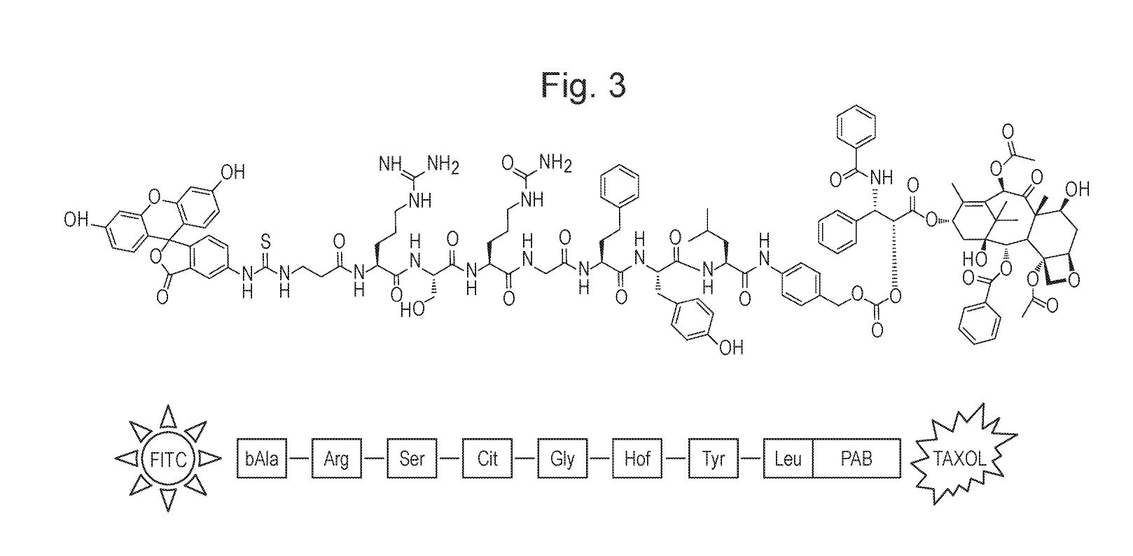

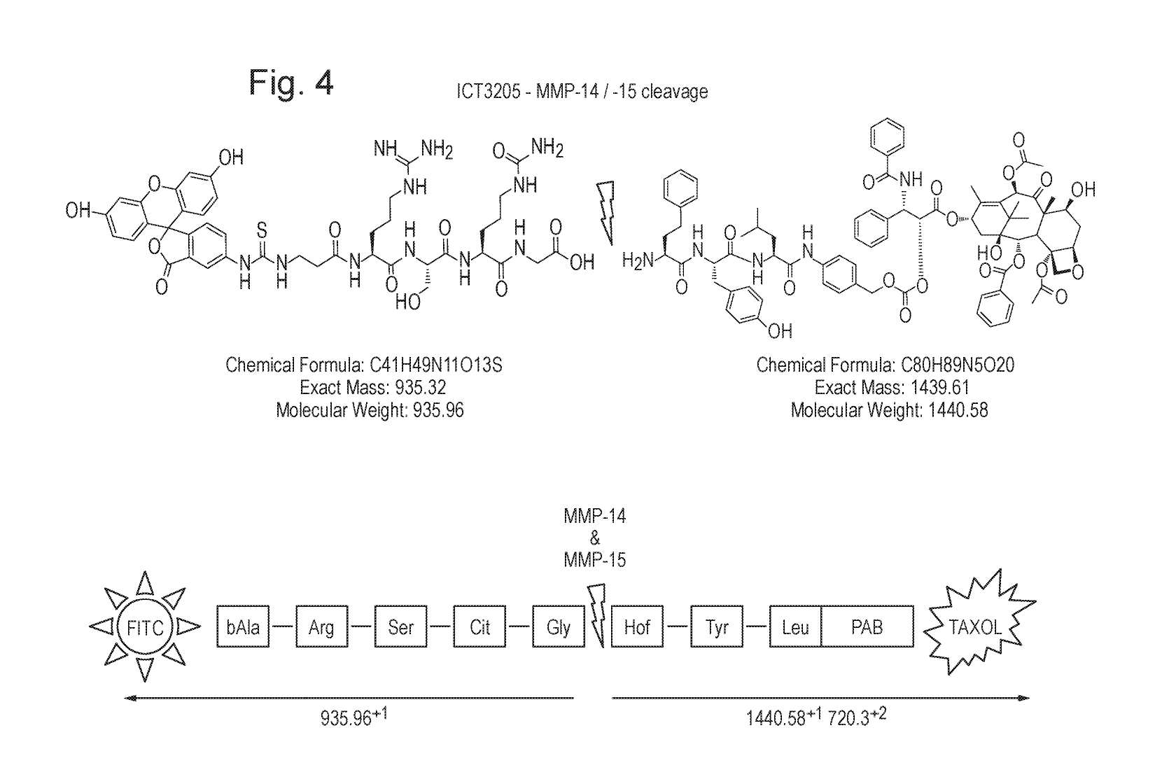

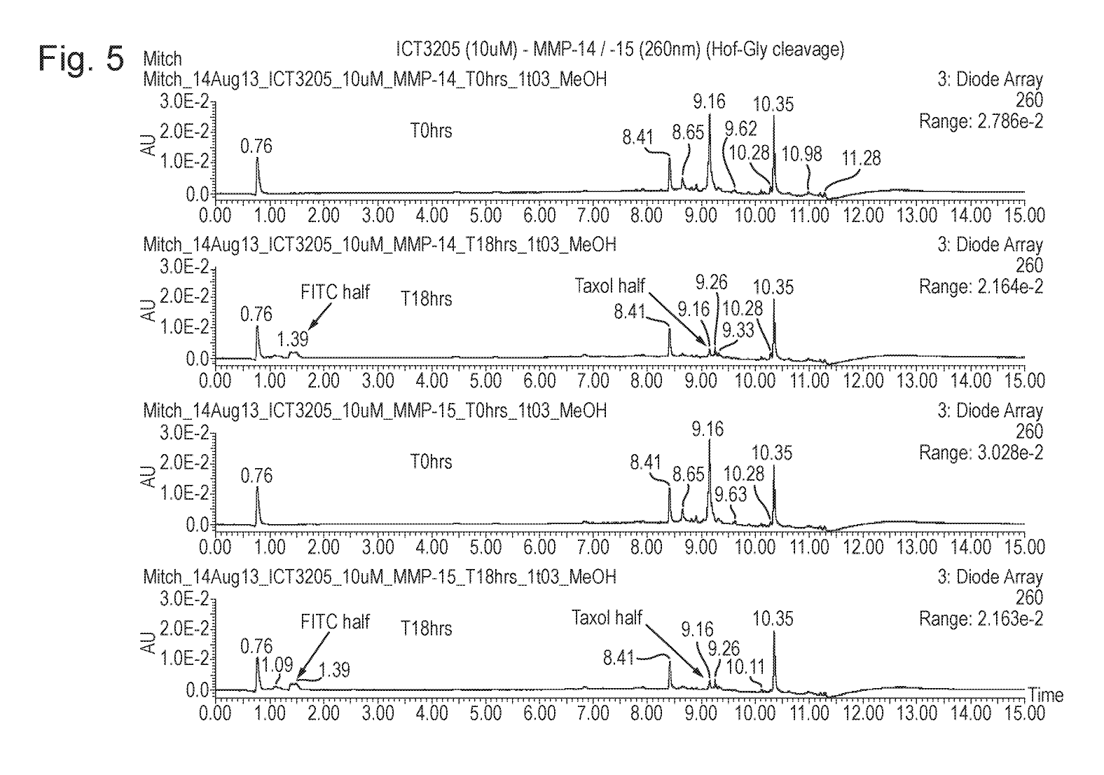

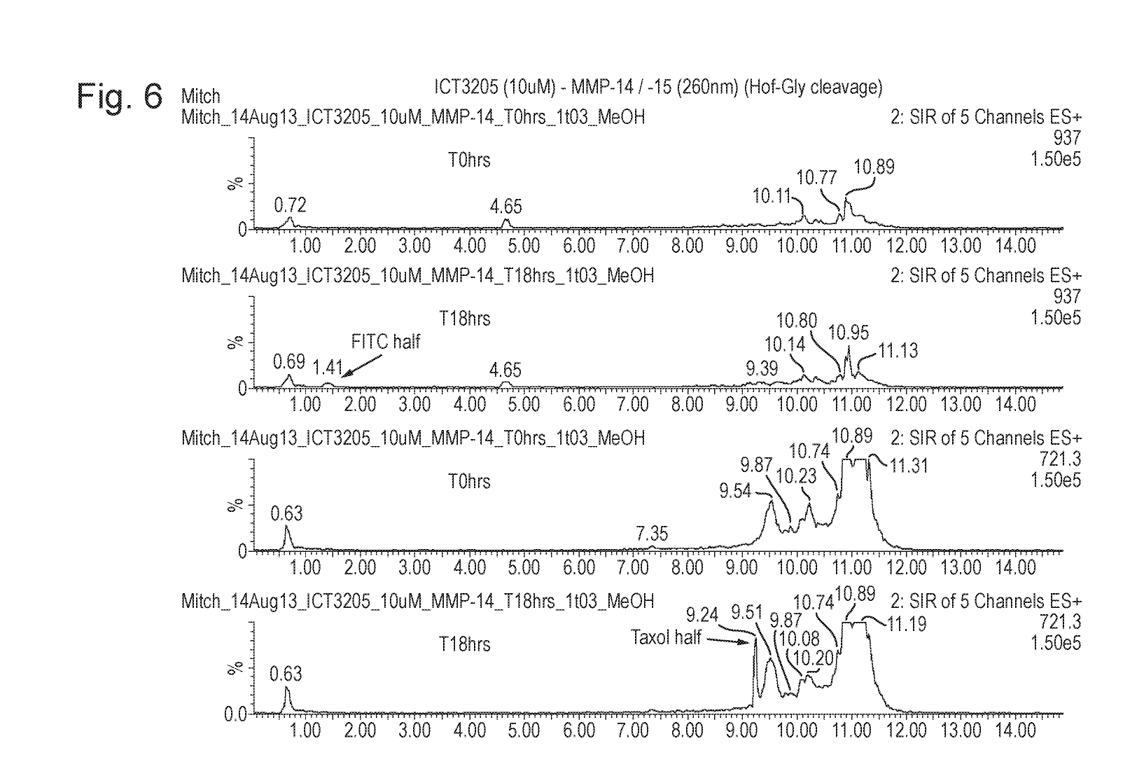

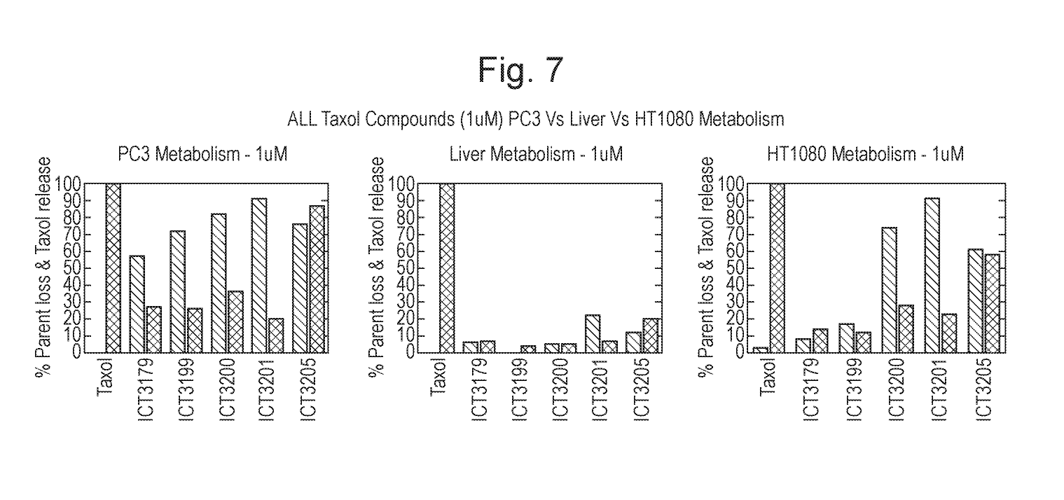

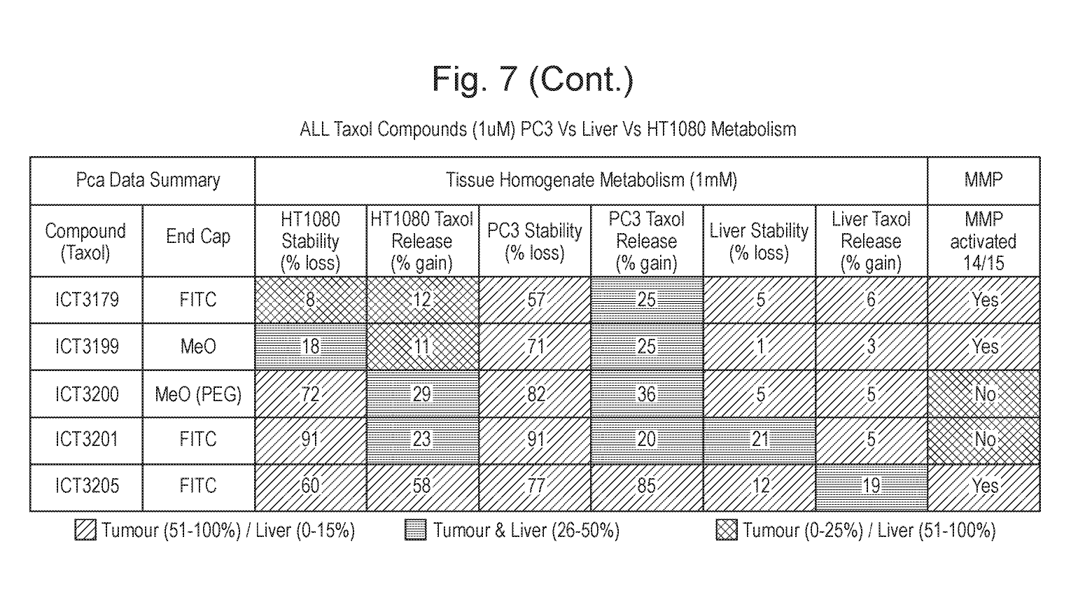

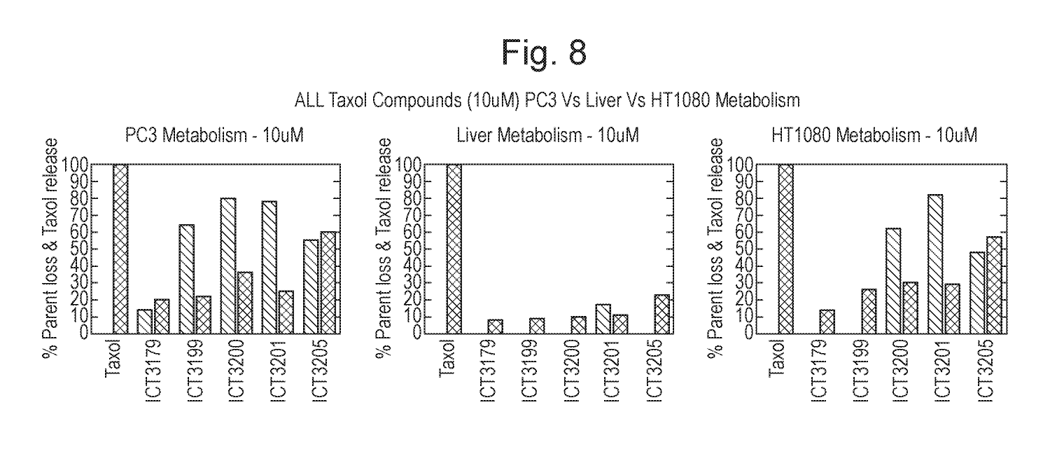

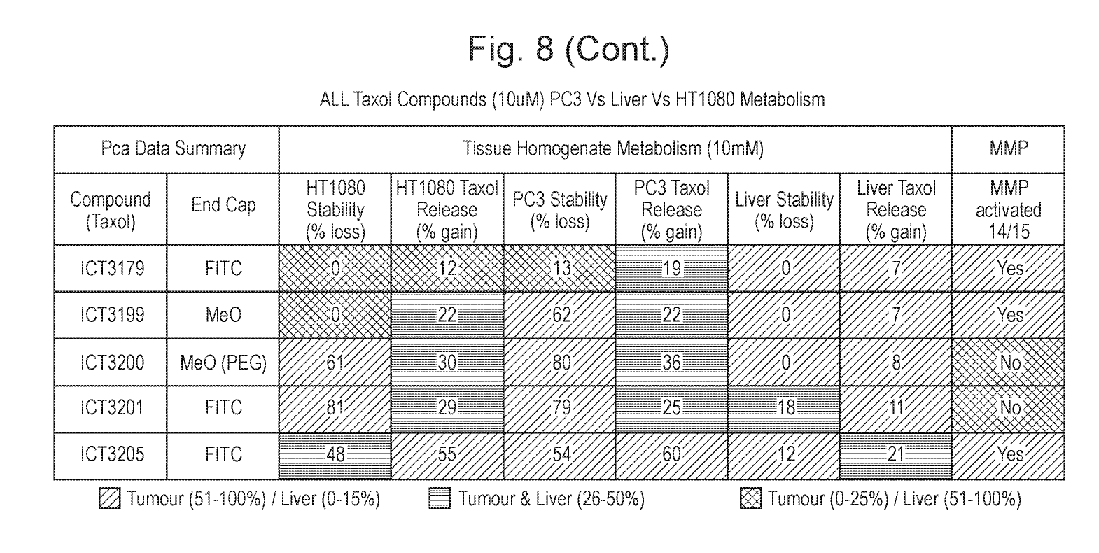

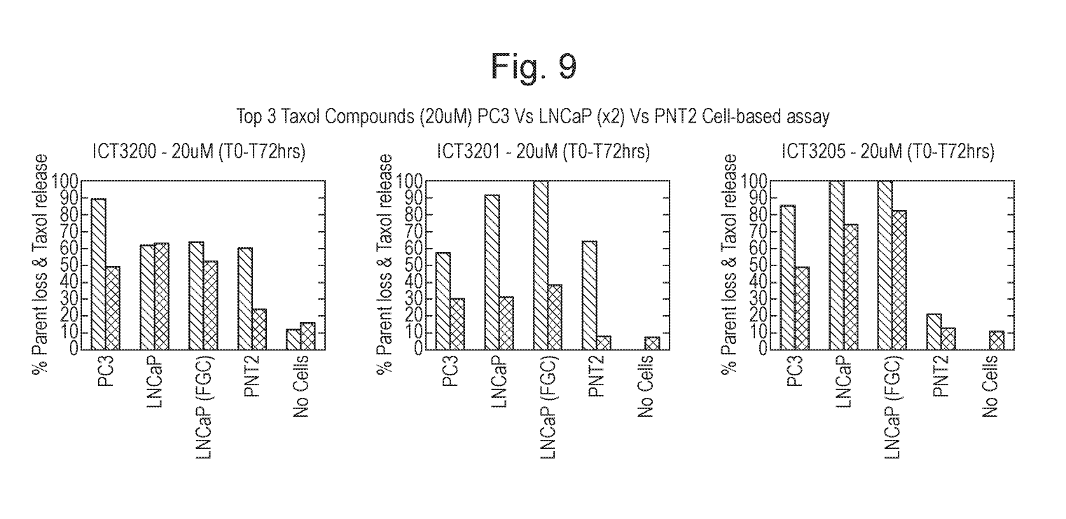

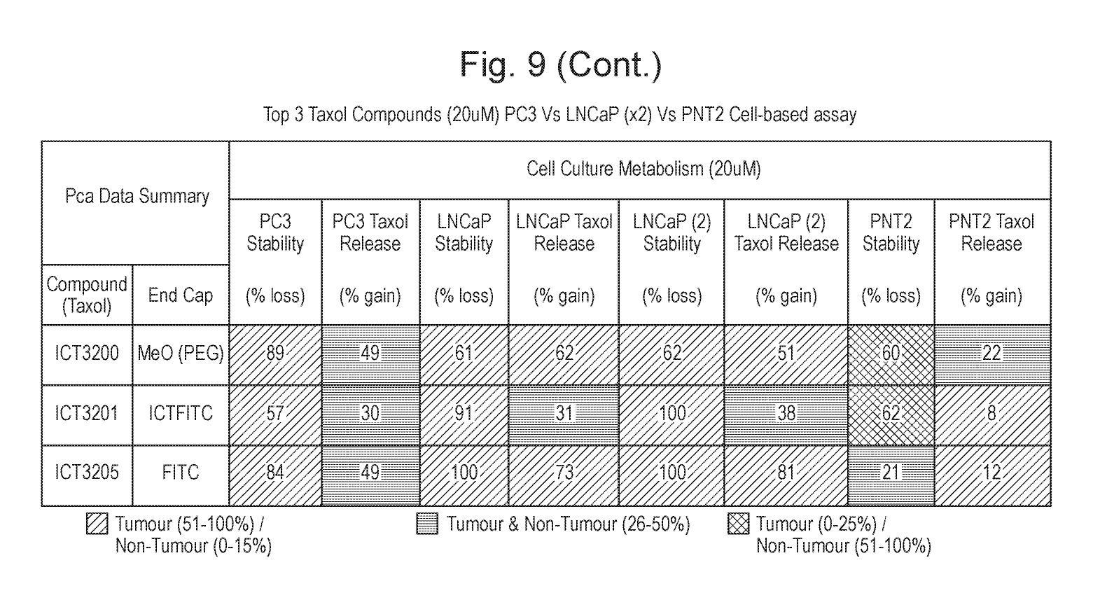

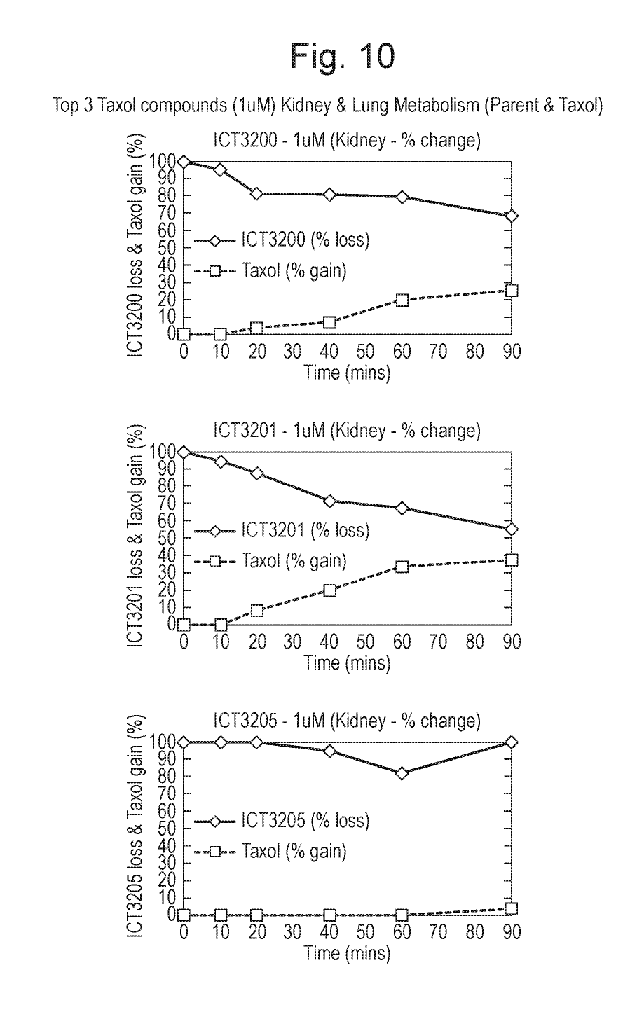

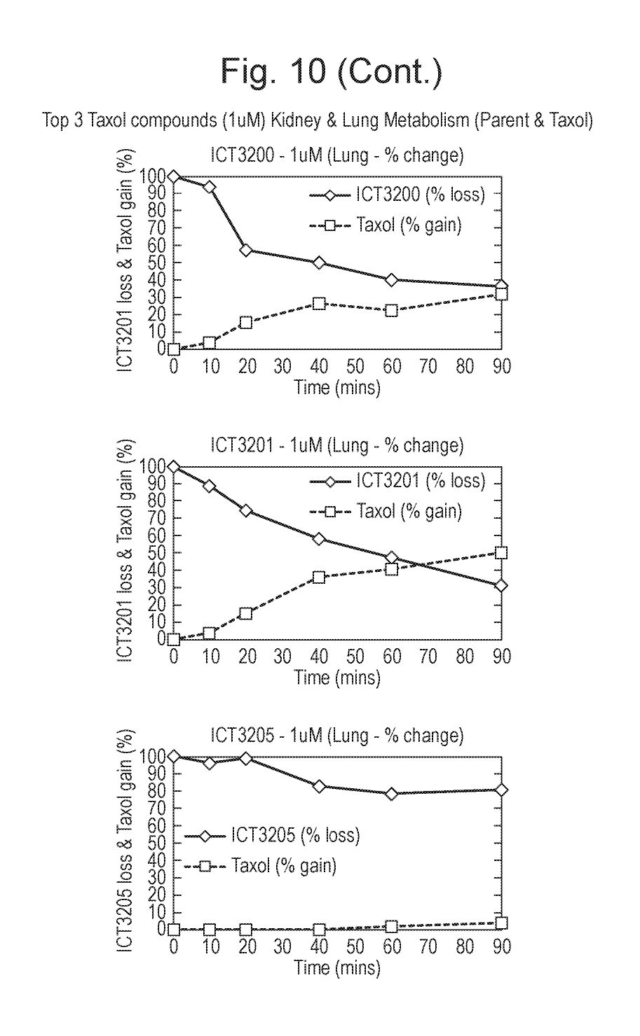

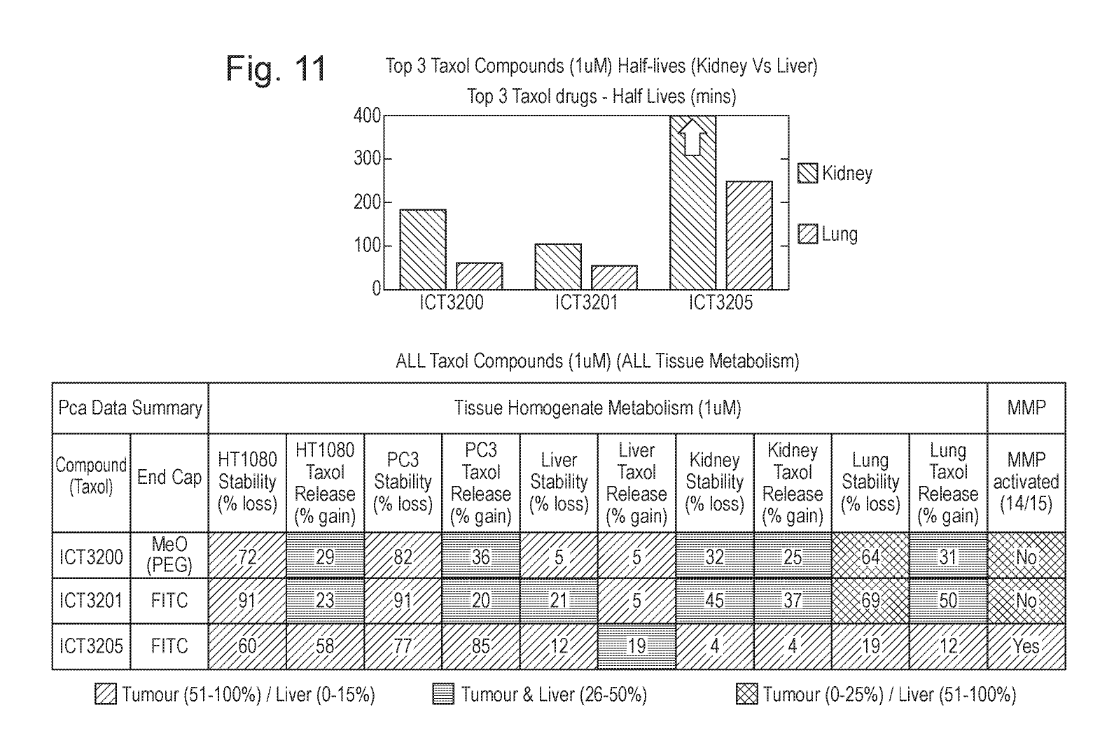

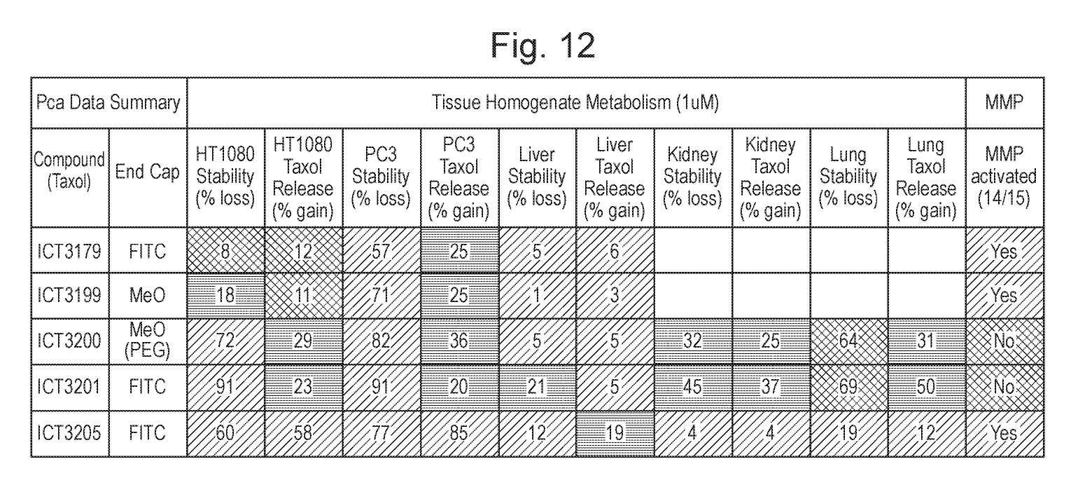

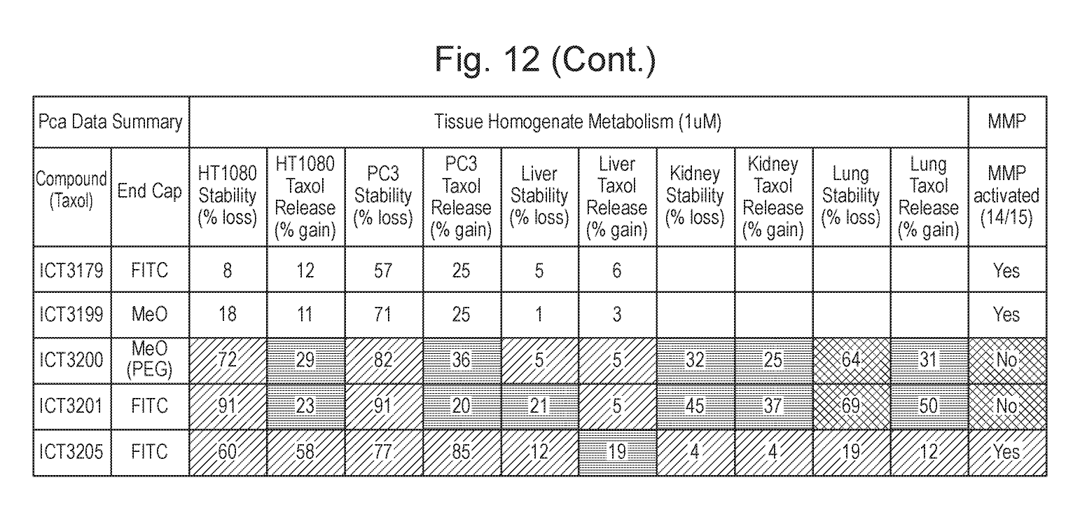

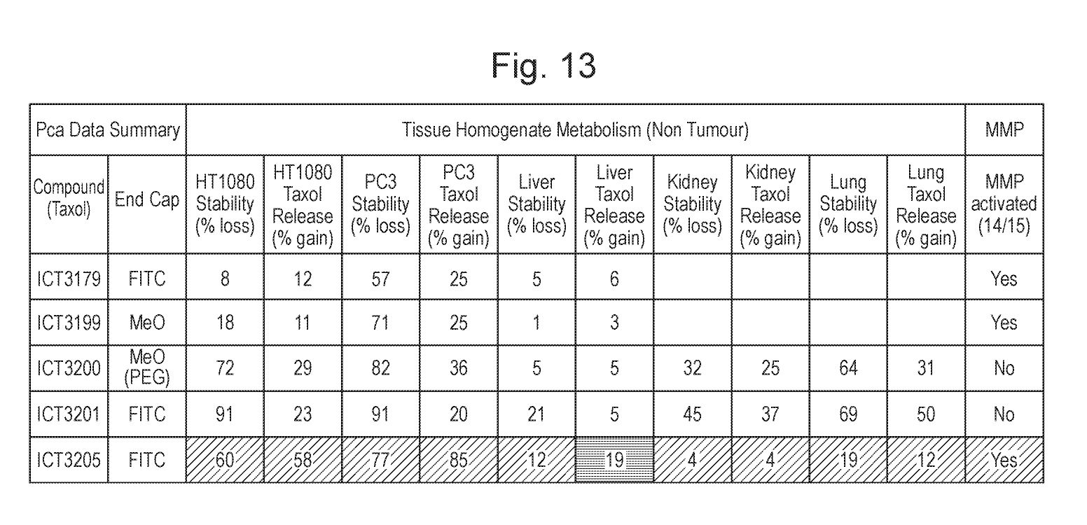

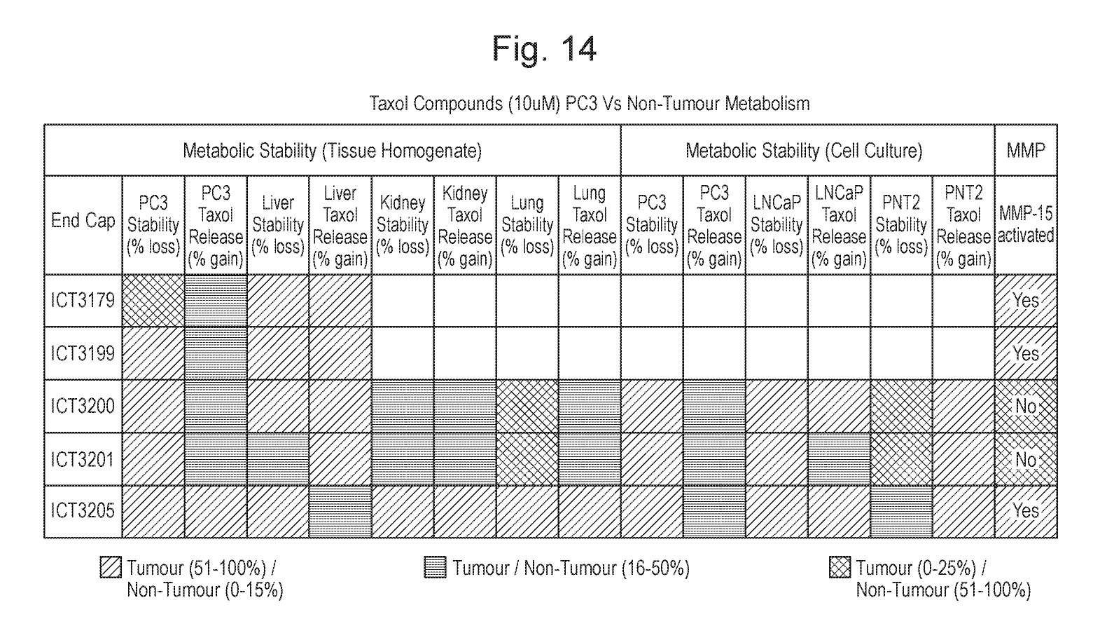

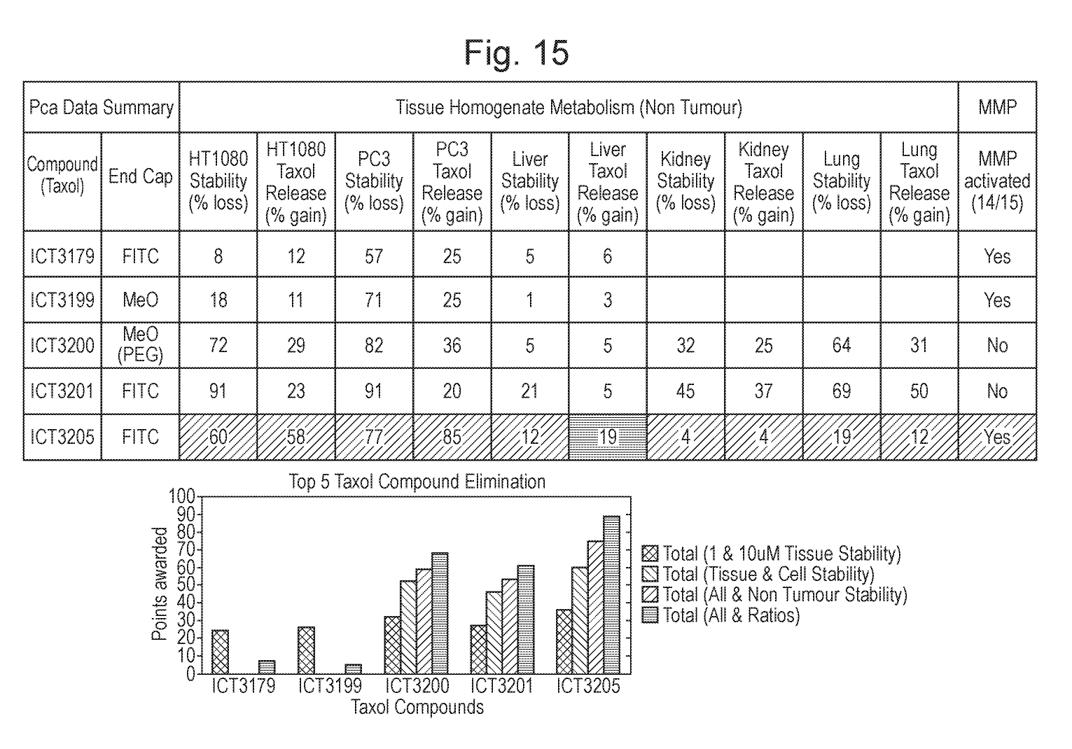

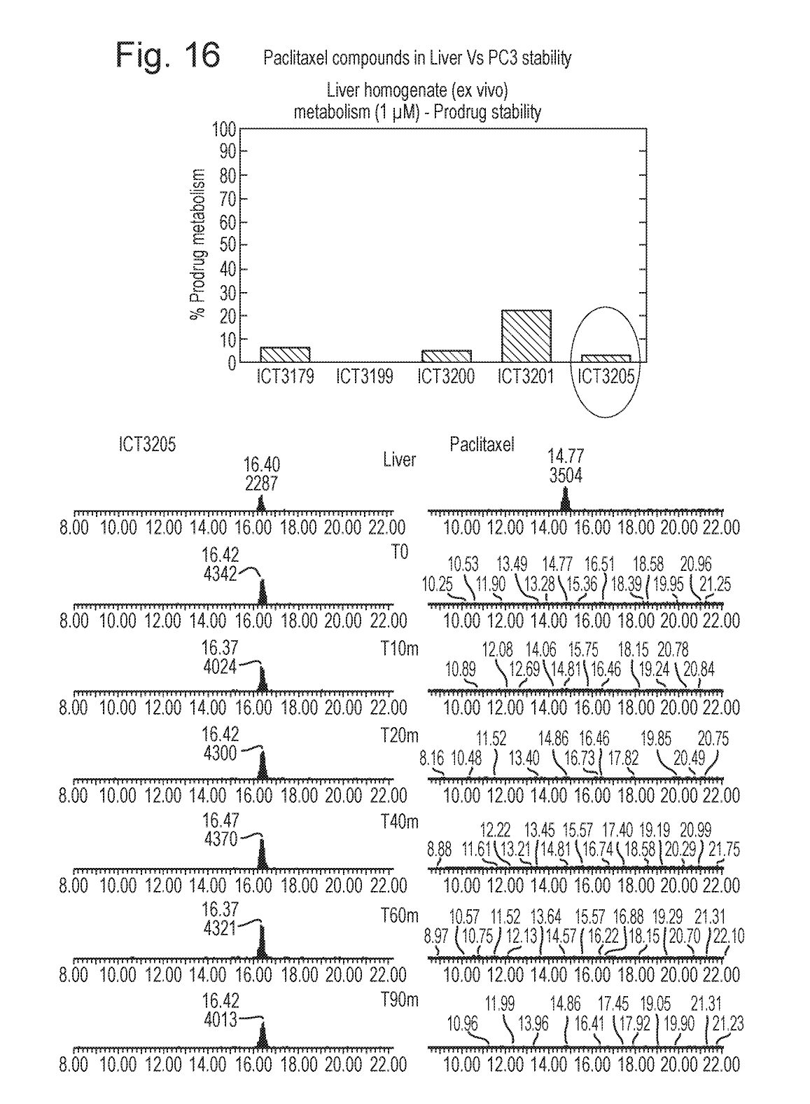

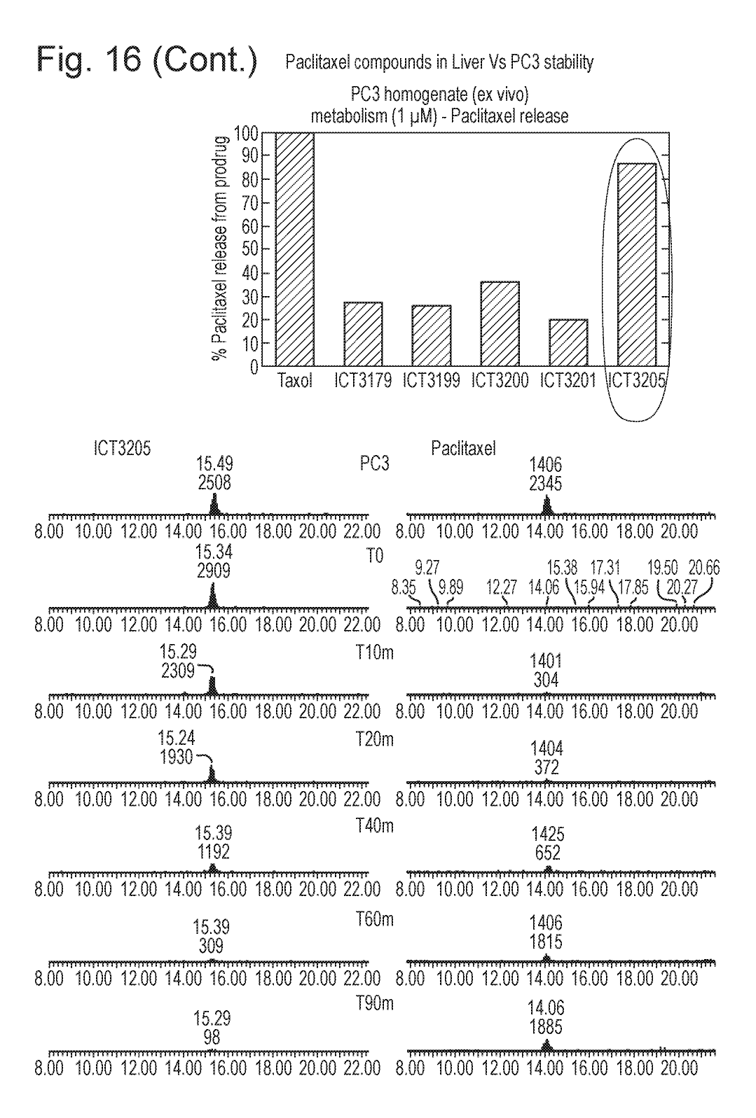

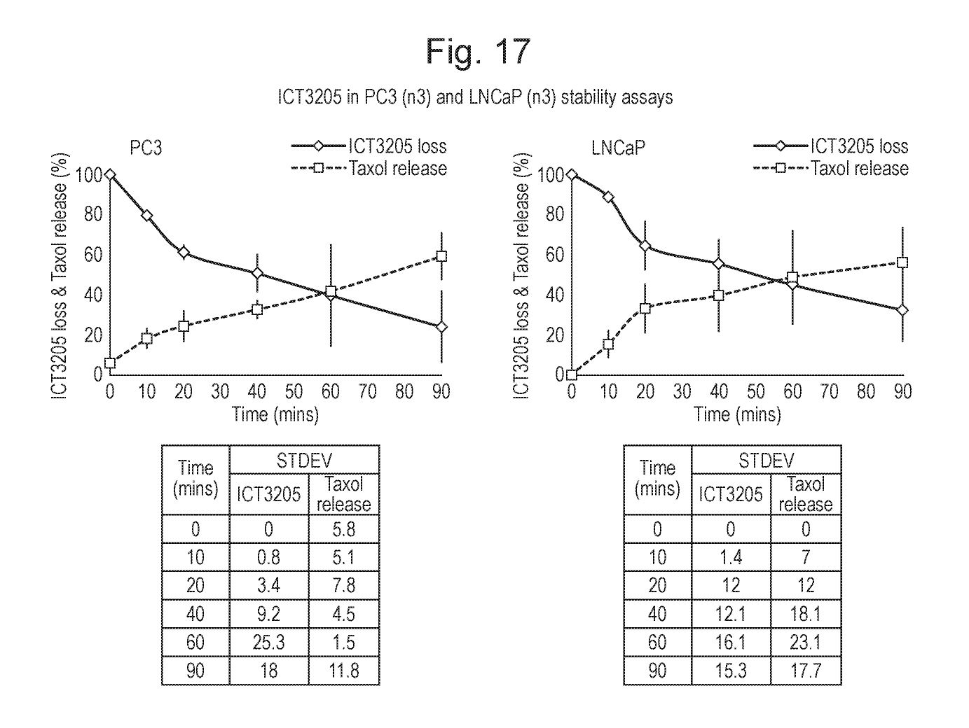

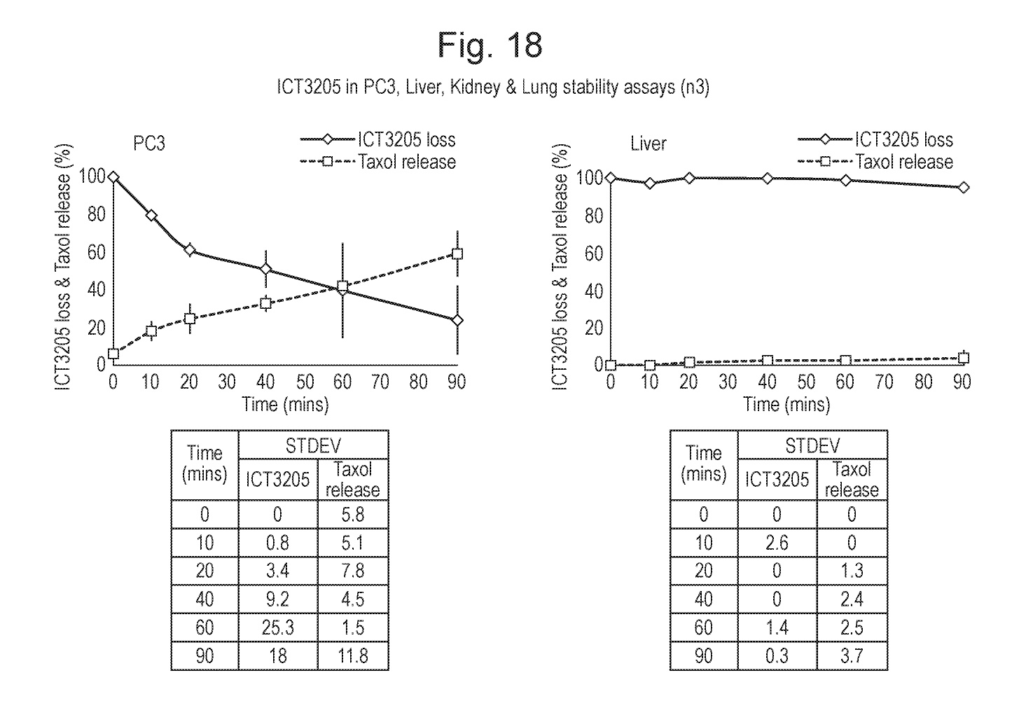

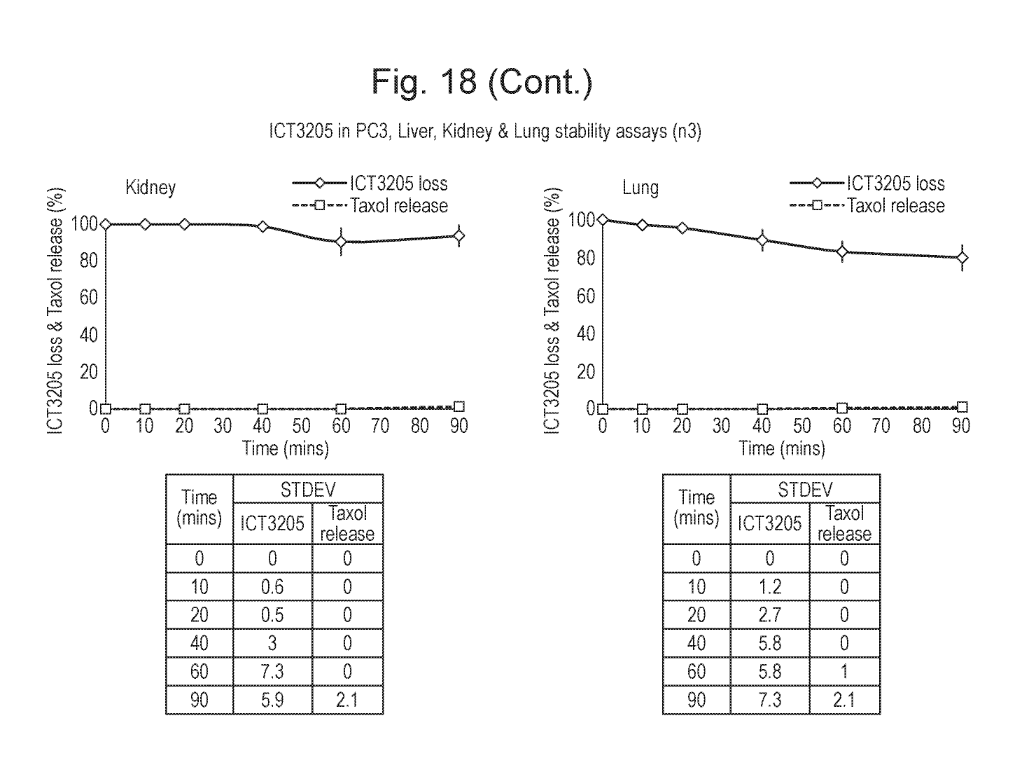

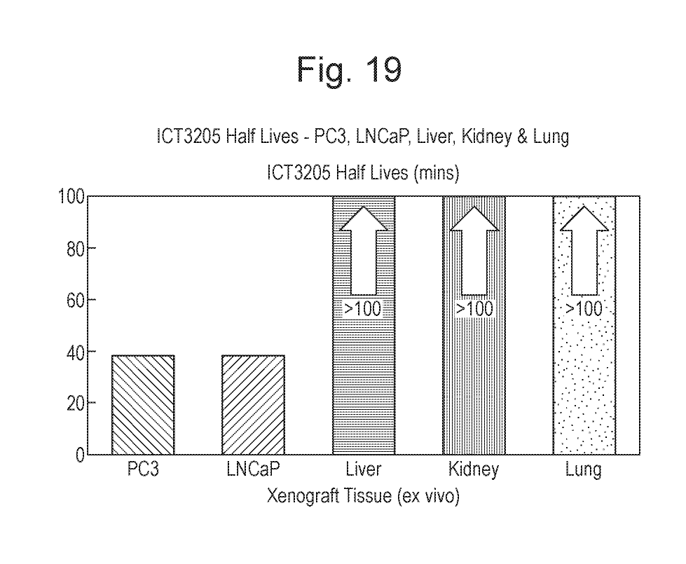

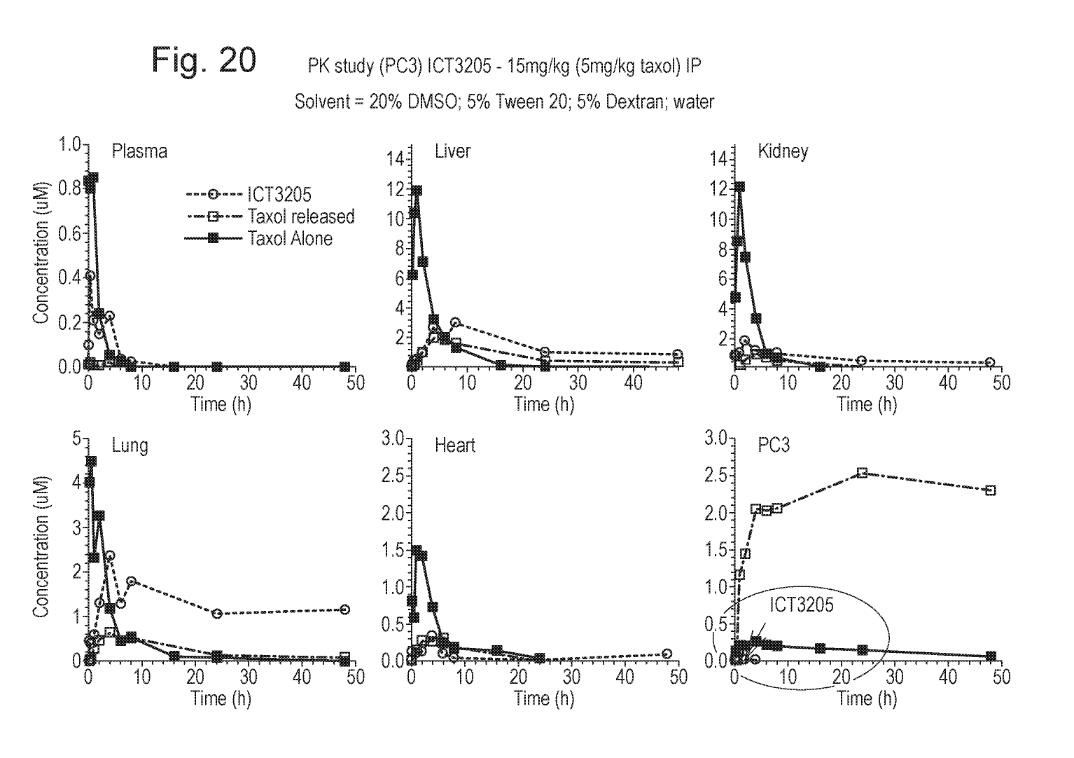

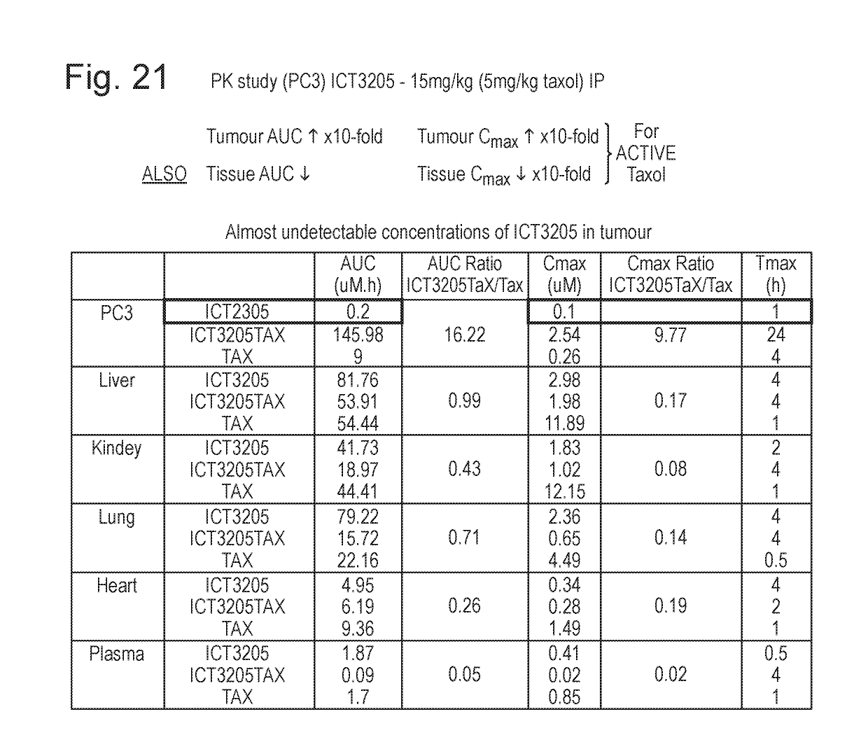

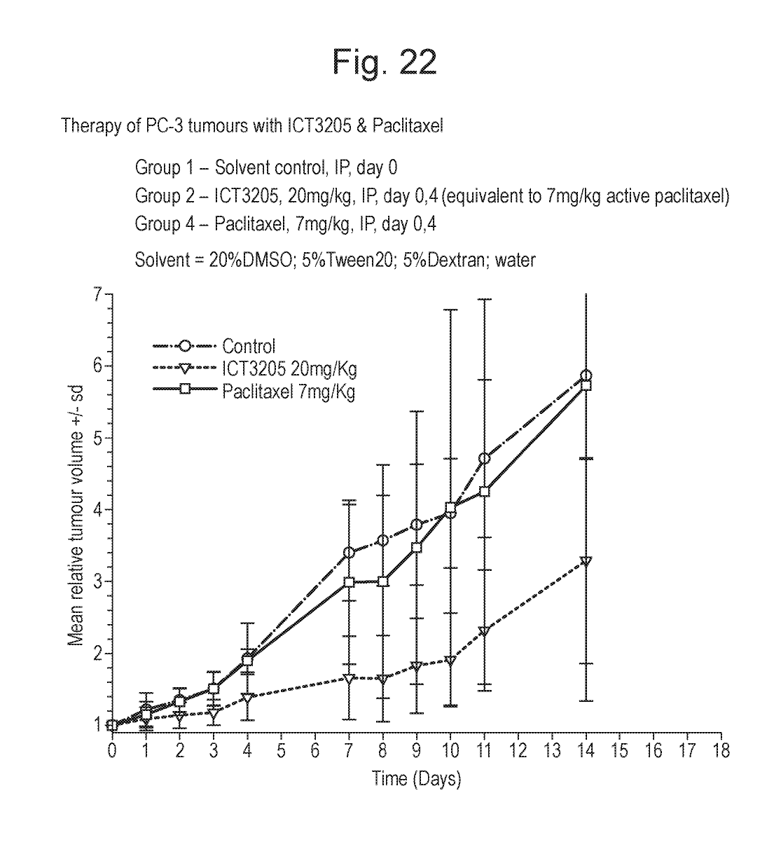

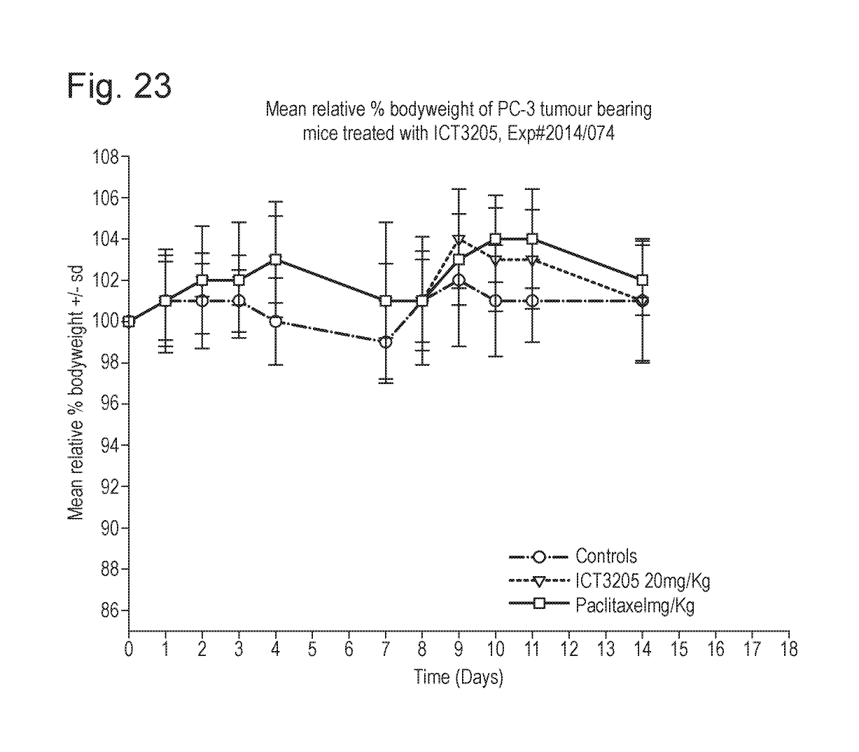

In a second aspect of the present invention the conjugate of a paclitaxel linked directly or indirectly via a self-immolative linker to a peptide comprising a MT-MMP cleavage site is optimized to select the conjugate demonstrating unexpectedly superior stability in non-tumour tissue and unexpectedly superior paclitaxel release kinetics. The second aspect of the present invention provides a conjugate comprising paclitaxel linked directly or indirectly via a PAB linker moiety to a peptide comprising a membrane type matrix metalloproteinase (MT-MMP) cleavage site having the amino acid sequence -Arg-Ser-Cit-Gly-Hof-Tyr-Leu-, and having an FITC capping group. This conjugate was called ICT3205 and the structure is shown in FIG. 3. Cleavage of ICT3205 by MMP-14 and/or MMP-15 is shown in FIG. 4.

Conjugate ICT3205 has been demonstrated to have systemic stability in normal tissues, but is rapidly cleaved to release cytotoxic paclitaxel at the tumour site or tumour microenvironment as demonstrated in the Figures.

Pharmaceutical Formulations

A third aspect of the present invention provides a pharmaceutical formulation comprising a conjugate of the present invention and a pharmaceutically acceptable carrier, diluent or excipient. A pharmaceutical formulation may contain pharmaceutically acceptable concentrations of salt, buffering agents, preservatives, compatible carriers and optionally other therapeutic agents.

Medical Uses

In a fourth aspect a conjugate of the present invention is used in therapy.

Conjugates of the present invention may be used to treat a disease or condition associated with elevated MT-MMP activity, and optionally elevated MMP-14 and/or MMP-15 activity. Elevated MT-MMP activity, and optionally elevated MMP-14 and/or MMP-15 activity is associated with cancer.

A conjugate of the present invention is for use in treating cancer. The present invention also provides a method of treating a cancer in a subject, comprising administering to the subject a therapeutically effective amount of a conjugate of the present invention.

Cancer is a malignancy of epithelial, endodermal or mesenchymal origin, such as a carcinoma or sarcoma. Carcinomas include cervix, prostate, breast, nose, head and neck, oral cavity, oesophagus, stomach, liver, pancreas, colon, ovary, urinary bladder or lung, preferably non-small cell lung carcinoma. Sarcomas include, bone, cartilage, adipose tissue, smooth muscle, skeletal muscle, nerve sheath, blood vessels, mesothelium and gastrointestinal stroma sarcoma.

In embodiments a conjugate of the present invention is for use in the treatment of prostate cancer.

The present invention provides a conjugate comprising paclitaxel linked directly or indirectly via a PAB linker moiety to a peptide comprising a membrane type matrix metalloproteinase (MT-MMP) cleavage site having the amino acid sequence -Arg-Ser-Cit-Gly-Hof-Tyr-Leu-, and having an FITC capping group, for use in a method of treatment prostate cancer.

### EXAMPLES

### Example 1

### Synthesis of a Paclitaxel-Peptide Conjugate

Synthesis of Fmoc-Leu-PAB-OH (1)

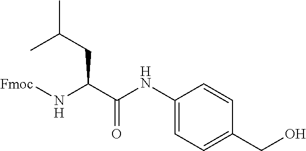

p-Aminobenzyl alcohol (PAB-OH) (310 mg, 2.59 mmol) was added to a solution of Fmoc-Leu-OH (830 mg, 2.35 mmol) and N-ethoxycarbonyl-2-ethoxy-1,2-dihydroquinoline (EEDQ) (630 mg, 2.59 mmol) in anhydrous dichloromethane (DCM) (50 mL) and the reaction solution was stirred at room temperature overnight. The solvent was then evaporated under reduced pressure and the residue was purified by column chromatography eluting with DCM:MeOH 98:2 to afford 1 (1.00 g, 92%) as a colourless powder.

¹H-NMR (400 MHz, CDCl₃): 0.83 (d, 3H, J=6.3 Hz); 0.85 (d, 3H, J=6.3 Hz); 1.55-1.69 (m, 3H); 4.00 (t, 1H, J=7.3 Hz); 4.17 (dd, 1H, J=10.1 Hz, J=7.3 Hz); 4.25 (dd, 1H, J=10.1 Hz, J=7.3 Hz); 4.34-4.40 (m, 1H); 4.41 (5, 2H); 5.90 (d, 1H, J=7.2 Hz); 6.98 (d, 1H, J=8.0 Hz); 7.13 (d, 1H, J=7.4 Hz); 7.14 (d, 1H, J=7.4 Hz); 7.23-7.30 (m, 4H); 7.39 (d, 2H, J=7.4 Hz); 7.44 (d, 2H, J=7.5 Hz); 7.63 (dd, 2H, J=7.5 Hz, J=3.1 Hz); 8.69 (bs, 1H).

¹³C-NMR (400 MHz, CDCl₃): 21.9, 22.9, 24.7, 41.2, 46.9, 54.3, 64.6, 67.3, 119.9, 120.0, 124.9, 125.0, 127.1, 127.5, 127.7, 136.7, 137.1, 143.5, 143.7, 156.9, 171.3.

MS (APCI, 5 IA,MeCN): miz 459.0 ([M+H]+, 100).

Synthesis of Fmoc-Leu-PAB-PNP (2)

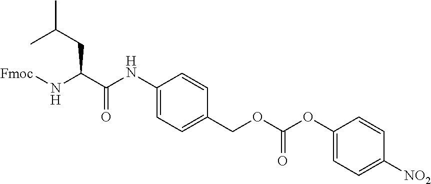

Pyridine (246 μL, 3.04 mmol) was added to a solution of 1 (700 mg, 1.52 mmol) and p-nitrophenyl chloroformate (PNPCI) (917 mg, 4.57 mmol) in anhydrous THF (30 mL) (and a few drops of anhydrous DMF if reqd to fully dissolve the solids), and the reaction was stirred for 30 minutes at room temperature. Subsequently, the solvent was evaporated under reduced pressure, and the residue was purified by column chromatography starting with DCM, and then adjusting the gradient to DCM:MeOH (100:3) to afford 2 (800 mg, 84%) as a colourless powder.

¹H-NMR (400 MHz, CDCl₃): 0.96 (d, 3H_{;} J=6.0 Hz); 0.98 (d, 3H, J=6.4 Hz); 1.62-1.81 (m, 3H); 4.18 (t, 1H, J=6.4 Hz); 4.35-4.48 (m, 3H); 5.22 (s, 2H); 5.61 (d, 1H, J=7.6 Hz); 7.22-7.43 (m, 8H); 7.49-7.59 (m, 4H); 7.77 (d, 2H, J=7.6 Hz); 8.28 (d, 2H, J=8.8 Hz); 8.69 (bs, 1H).

¹³C-NMR (400 MHz, CDCl₃): 22.9, 24.7, 40.8, 47.0, 54.3, 67.3, 70.5, 120.0, 120.0, 121.7, 124.9, 125.3, 127.1, 127.8, 129.6, 129.9, 138.4, 141.2, 143.4, 143.5, 145.3, 152.4, 155.4, 156.8, 170.9.

MS (ESI, 5 kV,MeCN): m/z 623.9 ([M+H]+, 100), 646.0 ([M+Na]+, 88).

Synthesis of Fmoc-Leu-PABC-PTX (3)

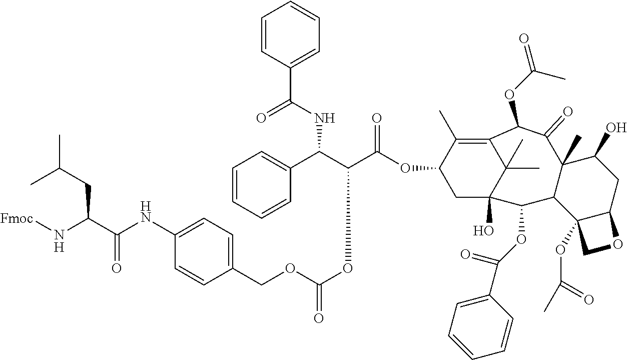

Paclitaxel (PTX) (370 mg, 440 μmol) and 4-(dimethylamino)pyridine (DMAP) (50.0 mg, 440 μmol) were added to a solution of 2 (270 mg, 440 μmol) in anhydrous DCM (15 mL), and the reaction was stirred at room temperature for 24 h. The solvent was evaporated under reduced pressure and the residue was purified by column chromatography, eluting with ethyl acetate:hexane 1:1 to afford 3 (550 mg, 93%) as a colourless powder.

¹H-NMR (400 MHz, CDCl₃): 0.94 (d, 3H, J=6.6 Hz); 0.95 (d, 3H, J=6.6 Hz); 1.14 (s, 3H); 1.22 (s, 3H); 1.69 (5, 3H); 1.85 (s, 3H); 2.20 (s, 3H); 2.37 (dd, 1H, J=15.4, J=9.4); 2.43 (s_{;} 3H); 2.51-2.60 (m, 1H); 3.79 (d, 1H, J=7.1); 4.18-4.23 (m, 2H); 4.31 (d, 1H, J=8.5); 4.39-4.50 (m, 3H); 4.97 (dd, 1H, J=9.5, J=1.7); 5.08 (d, 1H, J=11.9); 5.15 (d, 1H, J=11.9); 5.13 (d, 1H, J=11.7 Hz); 5.47 (d, 1H, J=3.0); 5.71 (d, 1H, J=6.8); 5.73 (d, 1H, J=8.1); 6.00 (dd, 1H, J=9.2, J=2.7); 6.27 (t, 1H, J=8.6); 6.33 (bs, 1H); 7.16 (d, 1H, J=9.1); 7.21-7.30 (m, 4H); 7.32-7.43 (m, 8H); 7.44-7.63 (m, 8H); 8.15 (dd, 2H, J=7.1, J=1.3); 8.75 (bs, 1H).

^{—}C-NMR (400 MHz, CDCl₃): 9.6, 14.6, 20.8, 21.9, 22.0, 22.6, 22,9. 24,6. 26.7, 35.5, 35.6, 40.8, 43.1, 45.6, 46.9, 52.7, 54.1, 58.3, 60.4, 67.1, 70.4, 71.9, 72.0, 75.0, 75.5., 76.3, 78.8, 80.9, 84.4, 115.5, 119.9, 120.0, 124.9, 126.5, 127.0, 127.1, 127.7, 128.5, 128.6, 128.6, 129.0, 129.1, 129.4, 130.0, 130.0, 130.1, 131.9, 132.8, 133.4, 133.6, 136.5, 138.2, 141.2, 142.2, 143.4, 143.5, 153.7, 156.7, 162.6, 166.8, 167.3, 167.8, 169.7, 171.0, 171.1, 203.6.

MS (ESI, 5 kV, MeOH): m/z 1360.1 ([M+Na]+, 100), 1338.0 ([M+H]+, 60).

Synthesis of H-Leu-PAB-PTX (4)

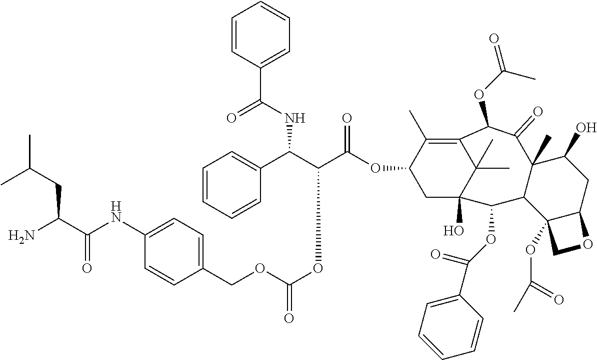

The Fmoc group was first removed from the compound by treatment of 3 (500 mg, 370 μL) with 1% DBU in THF (10 mL) at RT for 45 seconds. The reaction should be promptly quenched with a solution of HCI in ether. The solvents were then evaporated and the product was purified by column chromatography DCM:MeOH (95:5 to 9:1) to afford 4 (350 mg, 84%) as a colourless powder.

Synthesis of Peptide (5)

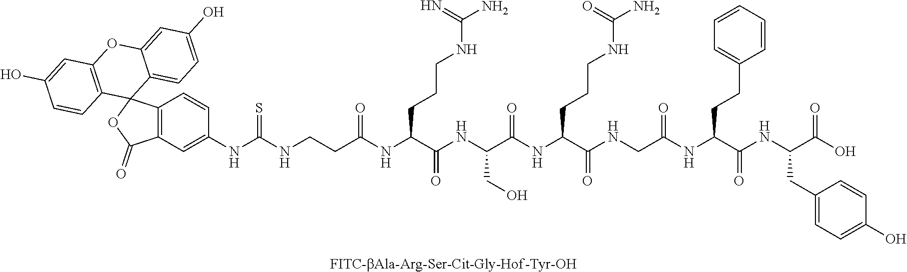 [SMILES: N=C(N)NCCC[C@H](NC(=O)CCNC(=S)Nc1ccc2c(c1)C(=O)OC21c2ccc(O)cc2Oc2cc(O)ccc21)C(=O)N[C@@H](CO)C(=O)N[C@@H](CCCNC(N)=O)C(=O)NCC(=O)N[C@@H](CCc1ccccc1)C(=O)N[C@@H](Cc1ccc(O)cc1)C(=O)O]

Peptide 5 was synthesised using conventional solid phase peptide synthesis, using an Fmoc-based strategy. Commercially-available Fmoc-Tyr-OH immobilised on a Wang resin was utilised. Synthesis of peptide acids was achieved manually. The resin was swelled thoroughly in DMF, followed by removal of the N-Fmoc protecting group by treatment with 20% v/v piperidine in DMF (3×3 min).

All subsequent couplings were performed in DMF, employing 2.5-fold molar excesses of N-Fmoc protected amino acids (with appropriate side-chain protecting groups), and activated using HCTU/HOBt/DiPEA. N-Fmoc de-protections were performed using 20% piperidine in DMF (3×3 min). The success of couplings and de-protections was monitored using the ninhydrin-based Kaiser test. Unsuccessful couplings were repeated. After the final N-Fmoc deprotection, the peptide chain was endcapped with fluorescein isothiocyanate (2.50 eq, in the presence of DiPEA, 1.50 eq), The success of this reaction was also monitored by the Kaiser test.

An additional β-alanine residue was incorporated into the sequence to overcome incompatability of the thiourea linkage and the acidic conditions of cleavage (the thiourea can rearrange, and the carbonyl carbon of the preceding amide bond can undergo nucleophilic attack by the sulphydryl-like function so formed. This leads to cleavage of the amide bond, with concomitant formation of a cyclic thiazolinone. The thiazolinone can undergo rearrangement in the presence of aqueous acid to form a thiohydantoin).

On completion of the sequence, the resin was washed (DMF, CH₂Cl₂, CH₂Cl₂/MeOH) and dried in vacuo over KOH to constant weight.

Peptides were cleaved from the resin by mild acidolysis using trifluoroacetic acid (TFA):H₂O:triisopropylsilane 95:2.5:2.5 for 4 h at room temperature, with simultaneous side-chain de-protection. Following cleavage, the TFA was removed under reduced pressure. The crude product was extracted into 95% aqueous acetic acid and lyophilised. The crude peptide was subsequently analysed using reversed phase HPLC and purified using preparative HPLC (purity >97%). Pure fractions were combined and lyophilised. Identity was confirmed by mass spectrometry.

Synthesis of paclitaxel-PAB-peptide conjugate (6)

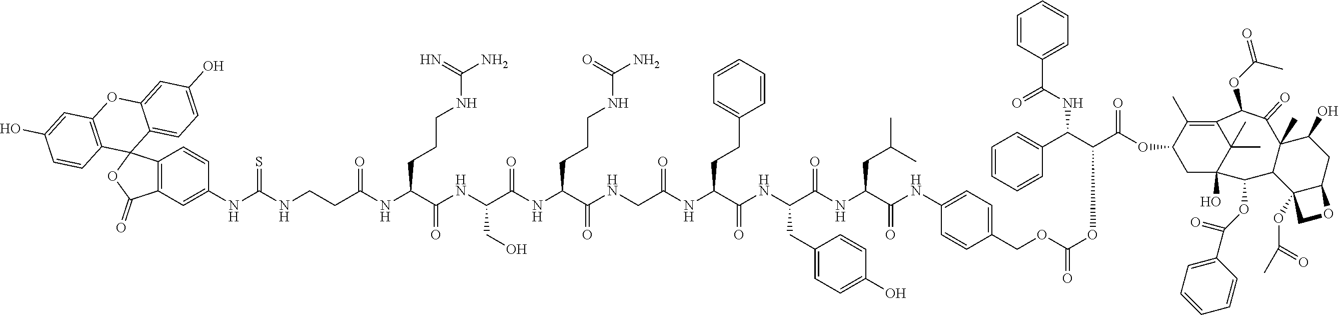

H-Leu-PAB-PTX 4 (22 mg, 0.020 mmol) was dissolved in anhydrous DMF (0.5 mL) and cooled to 0° C. To this solution peptide 5 (28 mg, 0.022 mmol, 1.1 equivalents) was added, along with hydroxybenzotriazole (HOBt) (8 mg, 0.079 mmol, 3 equivalents) and anhydrous N-methylmorpholine (NMM) (9 μL, 0.079 mmol, 4 equivalents) and the mixture was stirred for 15 minutes at 0° C. N,N′-Diisopropylcarbodiimide (DIC) (19 μL, 0.118 mmoles, 6 equivalents) was subsequently added and the reaction was stirred overnight at 4° C. Paclitaxel-PAB-peptide conjugate 6 was analysed (Rₜ=21 min; identity confirmed by LC-MS; methodology appended below) and purified by preparative HPLC.

Details of purification: the conjugate was purified on an Agilent 1100/1200 analytical HPLC system using an Agilent Zorbax XDB C18 column; 5 μM; 4.6×150 mm and a diode array detector @260 nm. Flow rate: 1.0 mL/min; Solvent A: H₂O+0.05% TFA; Solvent B: MeCN 90%, H₂O 10%+TFA 0.05%; a linear gradient of 30-100% B over 25 minutes was employed.

### Examples 2

Summary of Improved Delivery of Paclitaxel to Prostate Tumours: a Membrane-Type Matrix Metalloproteinase (MT-MMP) Targeted Approach.

Introduction: Membrane-type matrix metalloproteinases (MT-MMPs) are highly expressed and active in prostate tumours, but absent or inactive in normal tissues. MT-MMPs are also known to be elevated in the majority of solid human tumours and to be central to tumour invasion and angiogenesis. Our objective has been to design inactive prodrugs of paclitaxel that are converted to the active drug by selected MMPs within the prostate tumour microenvironment.

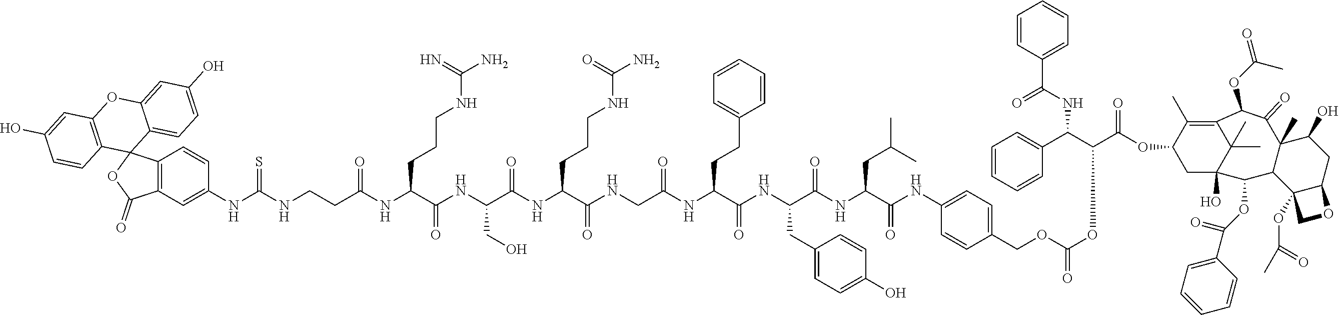

Fig A Structure of ICT3205 (See also FIG. 3)

Methods and Results: We report the synthesis and biological evaluation of a new series of peptide-based conjugates of paclitaxel designed to be selectively cleaved by MT-MMPs in the tumour microenvironment. Paclitaxel is conjugated to the peptide C-terminus via a self-immolative linker, while the N-terminus is protected from non-specific exopeptidase cleavage through the use of a masking group.

The relative importance of individual amino acids within the MT-MMP peptide recognition sequence has been investigated following extensive ex vivo metabolic studies. These studies employed tissue homogenates to assess activation of prodrug conjugates in tumour (PC-3) tissues and stability in normal tissues (liver kidney lung). ICT3205 (Fig A above) emerged as our lead agent, demonstrating in vitro stability in normal tissue with differential release of free paclitaxel in tumour tissue.

Further in vivo murine pharmacokinetic studies monitoring paclitaxel release in liver, lung, kidney heart and plasma revealed a substantial increase in tumour exposure to paclitaxel following ICT3205 prodrug administration (20 mg/kg) compared to the molar equivalent dose of paclitaxel alone (7 mg/kg). AUC (uM.h) ratios of paclitaxel released from ICT3205 compared to paclitaxel administered alone were 16.2 for tumour and in the range of 0.05-0.99 for normal tissues. Similar highly significant changes in were demonstrated with a 10-fold (9.77) increase in tumour concentrations and a substantially decreased Cₘₐₓ ratio (0.02-0.19) in other tissues when administered as ICT3205.

Anti-tumour efficacy studies (PC3 xenografts) resulted in a highly significant growth delay following single dose administration of ICT3205 [20 mg/kg] with paclitaxel alone (7 mg/kg; the molar equivalent dose to the paclitaxel released from ICT3205) having no significant anti-tumour effect

Conclusion: A series of peptide-based prodrug conjugates of paclitaxel were synthesized. 1CT3205 was identified as the lead molecule, enabling selective delivery of active paclitaxel to PC3 prostate tumours resulting in superior pharmacokinetics and efficacy when compared to delivery of paclitaxel alone.

1. A conjugate comprising a taxane linked directly or indirectly via a linker moiety to a peptide comprising a membrane type matrix metalloproteinase (MT-MMP) cleavage site having the amino acid sequence -Arg-Ser-aa1-Gly-Hof-aa2-aa3-, wherein each of aa1, aa2 and aa3 is any amino acid.

2. The conjugate according to claim 1, wherein the taxane is selected from paclitaxel, docetaxel or cabazitaxel.

3. The conjugate according to claim 1 or 2, wherein the taxane is paclitaxel.

4. The conjugate according to any one of claims 1 to 3, wherein the linker moiety is a self-immolative linker.

5. The conjugate according to claim 4, wherein the self-immolative linker is selected from para-amino benzoic acid (PAB),

6. The conjugate according to any one of the preceding claims, wherein aa1 is selected from Cit and Arg.

7. The conjugate according to any one of the preceding claims, wherein aa1 is Cit.

8. The conjugate according to any one of the preceding claims, wherein aa2 is selected from Tyr, Asp, Ala, Ser, Asn, Pro, Leu.

9. The conjugate according to any one of the preceding claims, wherein aa2 is Tyr.

10. The conjugate according to any one of the preceding claims, wherein aa3 is selected from Leu and Asn.

11. The conjugate according to any one of the preceding claims, wherein aa3 is Leu.

12. The conjugate according to any one of the preceding claims, wherein the MT-MMP cleavage site has the amino acid sequence
-Arg-Ser-Cit-Gly-Hof-Tyr-Leu-,
-Arg-Ser-Cit-Gly-Hof-Asn-Tyr-, or
-Arg-Ser-Arg-Gly-Hof-Tyr-Leu-.

13. The conjugate according to any one of the preceding claims, wherein the peptide comprises one or more amino acids at one or both ends of the MT-MMP cleavage site.

14. The conjugate according to any one of the preceding claims further comprising a capping group on the peptide to prevent non-specific degradation of the peptide.

15. The conjugate according to claim 5, wherein the capping group is selected from simple sugars, D-amino acids, proline imino acids, aromatics, aliphatics, fluorescein, or fluorescein derivatives, (e.g. that derived from fluorescein isothiocycante (FITC)), PEG and PEG derivatives.

16. The conjugate according to any one of the preceding claims further comprising a spacer between the peptide and the capping group.

17. The conjugate according to any one of the preceding claims further comprising a spacer between the linker moiety and the peptide.

18. The conjugate according to claim 16 or 17, wherein the spacer is selected from an amino acid, a non-natural amino acid, optionally beta-Ala, a succinyl group,

19. The conjugate according to any one of the preceding claims, wherein the taxane is linked directly or indirectly via a linker moiety to the C-terminus of the peptide.

20. The conjugate according to any one of the preceding claims, wherein the capping group is at the N-terminus of the peptide.

21. The conjugate according to any one of the preceding claims, wherein the conjugate comprises paclitaxel linked via PAB to the C-terminus of the peptide comprising -Arg-Ser-Cit-Gly-Hof-Tyr-Leu- and having the capping group FITC group at the N-terminus and having the spacer group beta-Ala between the peptide and the capping group.

22. A conjugate comprising paclitaxel linked directly or indirectly via a PAB linker moiety to a peptide comprising a membrane type matrix metalloproteinase (MT-MMP) cleavage site having the amino acid sequence -Arg-Ser-Cit-Gly-Hof-Tyr-Leu-, and having an FITC capping group.

23. A conjugate comprising Y-LX, wherein
Y is a peptide comprising a membrane type matrix metalloproteinase (MT-MMP) cleavage site having the amino acid sequence -Arg-Ser-aa1-Gly-Hof-aa2-aa3-,
wherein each of aa1, aa2 and aa3 is any amino acid,
L is a linker moiety, and
X is a taxane, each of Y, L and X as defined in any of claims 1 to 21.

24. A conjugate comprising C-Y-L-X, wherein
Y is a peptide comprising a membrane type matrix metalloproteinase (MT-MMP) cleavage site having the amino acid sequence -Arg-Ser-aa1-Gly-Hof-aa2-aa3-,
wherein each of aa1, aa2 and aa3 is any amino acid,
L is a linker moiety, and
X is a taxane,
C is a capping group, each of C, Y, L, X as defined in any of claims 1 to 21.

25. A conjugate comprising C-a-Y-L-X, wherein
Y is a peptide comprising a membrane type matrix metalloproteinase (MT-MMP) cleavage site having the amino acid sequence -Arg-Ser-aa1-Gly-Hof-aa2-aa3-,
wherein each of aa1, aa2 and aa3 is any amino acid,
L is a linker moiety, and
X is a taxane,
C is a capping group
a is a spacer, each of C, a, Y, L X as defined in claims 1 to 21.

26. A conjugate comprising C-a-Y-a-L-X, wherein
Y is a peptide comprising a membrane type matrix metalloproteinase (MT-MMP) cleavage site having the amino acid sequence -Arg-Ser-aa1-Gly-Hof-aa2-aa3-,
wherein each of aal aa2 and aa3 is any amino acid,
L is a linker moiety, and
X is a taxane,
C is a capping group
a is a spacer, each of C, a, Y, L X as defined in claims 1 to 21.

27. A pharmaceutical formulation comprising a conjugate according to any one of claims 1 to 26 and a pharmaceutically acceptable carrier, diluent or excipient.

28. The conjugate according to any one of claims 1 to 26 for use in medicine.

29. The conjugate according to any one of claims 1 to 26 for use in treating cancer.

30. The conjugate for the use of claim 29 wherein the cancer is a malignancy of epithelial, endodermal or mesenchymal origin, optionally wherein
a)the cancer is a carcinoma and preferably wherein the carcinoma is selected from cervix, prostate, breast, nose, head and neck, oral cavity, esophagus, stomach, liver, pancreas, colon, ovary, urinary bladder or lung, preferably non-small cell lung carcinoma, or
b)the cancer is a sarcoma, and preferably wherein the sarcoma is selected from bone, cartilage, adipose tissue, smooth muscle, skeletal muscle, nerve sheath, blood vessels, mesothelium and gastrointestinal stroma sarcoma.

31. The conjugate for the use of claim 29, wherein the cancer is prostate cancer.

32. A method of treating a cancer in a subject, comprising administering a therapeutically effective amount of conjugate according to claim 1 to a subject in need thereof.

33. A method according to claim 32, wherein the cancer is selected from the group consisting of a malignancy of epithelial, endodermal or mesenchymal origin, optionally wherein
a)the cancer is a carcinoma and preferably wherein the carcinoma is selected from cervix, prostate, breast, nose, head and neck, oral cavity, esophagus, stomach, liver, pancreas, colon, ovary, urinary bladder or lung, preferably non-small cell lung carcinoma, or
b)the cancer is a sarcoma, and preferably wherein the sarcoma is selected from bone, cartilage, adipose tissue, smooth muscle, skeletal muscle, nerve sheath, blood vessels, mesothelium and gastrointestinal stroma sarcoma.

34. A method according to claim 32, wherein the cancer is a prostate cancer.
